(19) United States
(12) Patent Application Publication
(10) Pub. No.: US 2025/0197942 A1
(43) Pub. Date: Jun. 19, 2025

(54) METHODS OF SELECTING AND TREATING CANCER SUBJECTS THAT ARE CANDIDATES FOR TREATMENT USING INHIBITORS OF A PD-1 PATHWAY

(71) Applicant: Arima Genomics, Inc., Carlsbad, CA (US)

(72) Inventor: Anthony Schmitt, Apex, NC (US)

(73) Assignee: Arima Genomics, Inc., Carlsbad, CA (US)

(21) Appl. No.: 18/844,261

(22) PCT Filed: Mar. 6, 2023
(86) PCT No.: PCT/US2023/014602

§ 371 Date: Sep. 5, 2024

Related U.S. Application Data
(60) Provisional application No. 63/400,867, filed on Aug. 25, 2022.
(60) Provisional application No. 63/317,377, filed on Mar. 7, 2022.

(51) Int. Cl. C12Q 1/6886; A61K 45/00

(52) U.S. Cl. CPC C12Q 1/6886; A61K 45/00; C12Q 2600/106; C12Q 2600/156

(57) ABSTRACT

The technology relates in part to methods of selecting for and/or treating subjects having cancer, where the subjects are identified as having at least one genetic structural variant that renders them suitable candidates for a treatment method that includes the administration of at least one inhibitor of a PD-1 pathway and/or an inhibitor of the interaction of the PD-1 receptor with PD-L1 and/or PD-L2.

### CROSS REFERENCE TO RELATED APPLICATIONS

This application claims the benefit under 35 U.S.C. § 119 (e) of U.S. provisional application No. 63/317,377, filed Mar. 7, 2022, and U.S. provisional application No. 63/400,867, filed Aug. 25, 2022. The entire contents of each of these referenced applications are incorporated by reference herein.

### FIELD

The technology relates in part to methods of selecting for and/or treating subjects having cancer, where the subjects are identified as having at least one genetic structural variant that renders them suitable candidates for a treatment method that includes the administration of at least one inhibitor of a PD-1 pathway.

### BACKGROUND

Cancers are often caused by genetic alterations, which include mutations (e.g., point mutations) and structural variations (e.g., translocations, inversions, insertions, deletions, and duplications). Genetic alterations can prevent certain genes from working properly. Genes that have mutations and/or structural variations that are linked to cancer may be referred to as cancer genes or oncogenes. Certain types of cancers have been linked to specific genetic alterations. However, there are cancers for which specific genetic alterations have not yet been identified.

A subject may acquire cancer-causing genetic alterations in a number of ways. In certain instances, a subject is born with a genetic alteration that is either inherited from a parent or arises during gestation. In certain instances, a subject is exposed to one or more factors that damage genetic material (e.g., UV light, cigarette smoke). In certain instances, genetic alterations arise as the subject ages. Given how cancer can cause cells to go haywire and replicate in an uncontrolled, invasive fashion, it is not unexpected that the same cancers often are caused by different genetic alterations, or by the synergistic effect of more than one genetic alteration.

Thus, the genetic make-up of subjects having cancer, even the same type of cancer, can vary widely, depending on differences in their genetic alterations. These differences in turn can lead to differences in their responsiveness to treatments. While the standard of care treatment may be effective in a majority of subjects having a particular type of cancer, e.g., triple-negative breast cancer or glioblastoma multiforme, certain subjects are not responsive or are less responsive to the standard of care treatment. For such subjects, there is a need to identify alternate treatments that can be administered in addition to, or instead of, the standard of care treatment. Provided herein are methods of selecting and/or treating cancer subjects (patients) that are suitable candidates for alternate treatment regimens based on the presence of one or more genetic alterations that render them responsive to an alternate treatment regimen.

### SUMMARY

Provided in certain aspects are methods of treating a subject that has, or is suspected of having, cancer, where the methods include: a) identifying and/or selecting a subject comprising a structural variant in the genome of the subject, where the location of the structural variant, or a breakpoint of the structural variant, is adjacent to the CD274 gene and/or the CD273 gene; and b) if the subject has cancer, treating the subject so identified and/or selected with a treatment that inhibits a PD-1 receptor-mediated pathway and/or inhibits the interaction of the PD-1 receptor with PD-L1 and/or PD-L2.

Also provided herein are methods of selecting a subject having cancer for treatment with an agent that inhibits the PD-1 receptor-mediated pathway and/or inhibits the interaction of a PD-1 receptor with PD-L1 and/or PD-L2, where the methods include: a) determining whether the subject comprises a structural variant in the genome of the subject, where the location of the structural variant, or a breakpoint of the structural variant, is adjacent to the CD274 gene and/or the CD273 gene; and b) if the structural variant, or a breakpoint of the structural variant, is identified in a), selecting the subject for treatment with an agent that inhibits a PD-1 receptor-mediated pathway and/or inhibits the interaction of the PD-1 receptor with PD-L1 and/or PD-L2.

Also provided herein are methods of screening a subject having cancer for potential responsiveness to treatment with an agent that inhibits a PD-1 receptor-mediated pathway and/or inhibits the interaction of the PD-1 receptor with PD-L1 and/or PD-L2, where the methods include: a) determining whether the subject comprises a structural variant in the genome of the subject, where the location of the structural variant, or a breakpoint of the structural variant, is adjacent to the CD274 gene and/or the CD273 gene; and b) if the structural variant, or a breakpoint of the structural variant, is identified in a), identifying the subject as a candidate for, and/or as potentially responsive to, treatment with an agent that inhibits a PD-1 receptor-mediated pathway and/or inhibits the interaction of the PD-1 receptor with PD-L1 and/or PD-L2.

In certain aspects of any of the methods provided herein, a breakpoint of the structural variant maps to a location between positions 5,471,000 to 5,480,000 on chromosome 9, where the positions are in reference to an HG38 human reference genome. In aspects, a breakpoint of the structural variant maps to a location between positions 5,475,001 to 5,476,000 on chromosome 9, where the positions are in reference to an HG38 human reference genome.

In certain aspects of any of the methods provided herein, the adjacent location of the structural variant and/or a breakpoint of the structural variant, is at a distance of about 4,000 base pairs from the CD274 gene, where the distance is measured from the 3′ end of the CD274 gene. In aspects, the adjacent location of the structural variant and/or a breakpoint of the structural variant is at a distance of about 4,400 base pairs from the CD274 gene, where the distance is measured from the 3′ end of the CD274 gene. In aspects, the adjacent location of the structural variant and/or a breakpoint of the structural variant is at a distance of between about 4,000 base pairs to about 30,000 base pairs from the CD274 gene, where the closest distance of about 4000 base pairs is measured from the 3′ end of the CD274 gene and the farthest distance of about 30,000 base pairs is measured from the 5′ end of the CD274 gene. In aspects, the adjacent location of the structural variant and/or a breakpoint of the structural variant is at a distance of between about 4,400 base pairs to about 25,000 base pairs from the CD274 gene, where the closest distance of about 4,400 base pairs is measured from the 3′ end of the CD274 gene and the farthest distance of about 25,000 base pairs is measured from the 5′ end of the CD274 gene. In certain aspects of any of the methods provided herein, the adjacent location of the structural variant and/or a breakpoint of the structural variant is at a distance of about 4,447 base pairs from the CD274 gene, where the distance is measured from the 3′ end of the CD274 gene. In aspects, the adjacent location of the structural variant and/or a breakpoint of the structural variant is at a distance of between a closest distance of about 4,447 base pairs, where the distance is measured from the 3′ end of the CD274 gene, to a farthest distance of about 24,459 base pairs from the CD274 gene, where the distance is measured from the 5′ end of the CD274 gene. In aspects, the distance is a linear distance.

In certain aspects of any of the methods provided herein, the adjacent location of the structural variant and/or a breakpoint of the structural variant, is at a distance of about 30,000 base pairs to about 35,000 base pairs from the CD273 gene, where the distance is measured from the 5′ end of the CD273 gene. In aspects, the adjacent location of the structural variant is at a distance of about 30,000 base pairs, 31,000 base pairs, 32,000 base pairs, 33,000 base pairs, 34,000 base pairs or 35,000 base pairs from the CD273 gene, where the distance is measured from the 5′ end of the CD273 gene. In aspects, the adjacent location of the structural variant and/or a breakpoint of the structural variant is at a closest distance of between about 33,000 base pairs to about 35,000 base pairs from the CD273 gene. In aspects, the adjacent location of the structural variant and/or a breakpoint of the structural variant is at a closest distance of about 34,531 base pairs from the CD273 gene. In certain aspects, the distance is a linear distance.

In certain aspects of any of the methods provided herein, the structural variant and/or a breakpoint of the structural variant includes an ectopic portion of genomic DNA from positions 18,377,000 to 18,386,000 on chromosome 9. In aspects, the structural variant and/or a breakpoint of the structural variant comprises an ectopic portion of genomic DNA from positions 18,381,001 to 18,382,000 on chromosome 9.

In aspects of any of the methods provided herein, the adjacent location is on the p arm of chromosome 9. In certain aspects, the ectopic portion of genomic DNA is on the p arm of chromosome 9.

Also provided herein are methods for treating a brain cancer that include administering a PD-1 receptor-mediated pathway inhibitor to a subject in need thereof in an amount effective for treating the brain cancer. In certain aspects, the cancer is a glioma. In aspects, the cancer is a glioblastoma. In certain aspects, the cancer is glioblastoma multiforme. In certain aspects, the cancer is pediatric glioblastoma. In aspects, the glioblastoma is a newly diagnosed glioblastoma. In certain aspects, the glioblastoma is a recurrent glioblastoma. In aspects, the subject is a pediatric subject.

In certain aspects of any of the methods provided herein, the subject is refractory to a standard of care treatment for cancer. In aspects, the cancer is a glioblastoma and the standard of care treatment can include performing and/or administering one or more of the following: surgical tumor resection, radiotherapy, positron emission tomography (PET)-guided radiotherapy, positron emission tomography (PET)-guided dose escalated radiotherapy, laser interstitial thermal therapy, stereotactic radiosurgery (SRS), hypofractionated stereotactic radiotherapy (HFSRT), Tumor-Treating Fields (TTFields), chemoradiotherapy, brachytherapy, carmustine implantable wafers, temozolomide, vincristine, interferon, bevacizumab, onartuzumab, a nitrosourea, procarbazine, enzastaurin, teniposide, cytarabine, vincristine, irinotecan, carboplatin, dasatinib, temsirolimus, erlotinib, sorafenib, veliparib, galunisertib, cediranib, vorinostat, panobinostat, dianhydrogalactitol (VAL-083) and paclitaxel poliglumex (PPX).

In certain aspects of any of the methods provided herein, the treatment that inhibits a PD-1 receptor-mediated pathway and/or inhibits the interaction of the PD-1 receptor with PD-L1 and/or PD-L2, which is administered in addition to or instead of the standard of care treatment, is selected from a treatment comprising administering one or more of atezolizumab, avelumab, balstilimab, cemiplimab, cemiplimab-rwlc, dostarlimab, dostarlimab-gxly, durvalumab, nivolumab, pembrolizumab, penpulimab, retifanlimab, sintilimab, pidilizumab, BMS-936559 (MDX-1105), AMP-224 fusion protein and MPDL33280A.

### BRIEF DESCRIPTION OF THE DRAWINGS

The drawings illustrate certain implementations of the technology and are not limiting. For clarity and ease of illustration, the drawings are not made to scale and, in some instances, various aspects may be shown exaggerated or enlarged to facilitate an understanding of particular implementations.

FIG. 1 shows an immunohistochemistry stain using anti-PD-L1 antibody in glioblastoma tumor cells.

Certain implementations are described further in the following description, examples and claims.

### DETAILED DESCRIPTION

Provided herein are methods of treating a subject that has, or is suspected of having, cancer, where the methods include identifying and/or selecting a subject comprising a structural variant in the genome of the subject, where the structural variant and/or a breakpoint of the structural variant is adjacent to the CD274 gene (also referred to interchangeably herein as the PD-L1 gene or the PDL-1 gene), which encodes the PD-L1 ligand (referred to interchangeably herein as PDL-1 or PD-L1). Absent an inversion, insertion, translocation, duplication or the like, or unless otherwise indicated, an “adjacent location” of a structural variant, or a breakpoint thereof, is in reference to the CD274 gene located on the “p” arm of chromosome 9, with coordinate positions between about 5,450,542 (5′-end) and about 5,470,554 (3′-end).

Also provided herein are methods of treating a subject that has, or is suspected of having, cancer, where the methods include identifying and/or selecting a subject comprising a structural variant in the genome of the subject, where the structural variant and/or a breakpoint of the structural variant is adjacent to the CD273 gene (also referred to interchangeably herein as the PD-L2 gene or the PDL-2 gene), which encodes the PD-L2 ligand (referred to interchangeably herein as PDL-2 or PD-L2). Absent an inversion, insertion, translocation, duplication or the like, or unless otherwise indicated, an “adjacent location” of a structural variant, or a breakpoint thereof, is in reference to the CD273 gene located on the “p” arm of chromosome 9, with coordinate positions between about 5,510,531 (5′-end) and about 5,571,282 (3′-end).

In aspects of the methods provided herein, the genome of the subject comprises a structural variant adjacent to the CD274 gene. In certain aspects of the methods provided herein, the genome of the subject comprises a structural variant adjacent to the CD273 gene. In aspects of the methods provided herein, the genome of the subject comprises a structural variant adjacent to the CD274 gene and comprises a structural variant adjacent to the CD273 gene.

Also provided herein are methods of selecting a subject having cancer for treatment with an agent that inhibits a PD-1 receptor-mediated pathway and/or inhibits the interaction of the PD-1 receptor with the PD-L1 and/or PD-L2 ligands. Also provided herein are methods of screening a subject having cancer for treatment with, and/or potential responsiveness to treatment with, an agent that inhibits a PD-1 receptor-mediated pathway and/or inhibits the interaction of the PD-1 receptor with the PD-L1 and/or PD-L2 ligands.

The terms “adjacent” or “outside of,” as used herein in reference to a structural variant being outside of or adjacent to a gene, such as the CD274 gene or the CD273 gene, generally means that a breakpoint of a structural variant is not within the CD274 gene or the CD273 gene. The structural variant can contain the gene, such as an inversion of the gene, an insertion of the gene, a duplication of the gene, or the like, or can contain a portion of the gene. In certain aspects, the structural variant may not include the CD274 gene, i.e., the structural variant does not contain the CD274 gene, insertion, inversion, duplication or any portion thereof. In certain aspects, the structural variant may not include the CD273 gene, i.e., the structural variant does not contain the CD273 gene, insertion, inversion, duplication or any portion thereof.

Adjacent generally means that the breakpoint of the structural variant is at a position or an equivalent distance that is between about 0 base pairs to about 1 Mb from the 5′ end or the 3′ end of the CD274 gene and/or the CD273 gene, such as about 0, 10,00, 20,000, 30,00, 40,000, 50,000, 50,000, 70,000, 80,000, 90,000, 100,000, 125,000, 150,000, 175,000, 200,000, 250,000, 300,000, 350,000, 400,000, 450,000, 500,000, 550,000, 600,000, 650,000, 700,000, 750,000, 800,000, 850,000, 900,000, 950,000, 975,000, 999,999 or more base pairs. In some embodiments, the breakpoint of the structural variant is at a position or an equivalent distance that is between about 0 base pairs to about 50,000 bp from the 5′ end or the 3′ end of the CD274 gene and/or the CD273 gene such as about 0, 100, 200, 300, 400, 500, 600, 700, 800, 900, 1000, 1500, 2000, 2500, 3000, 3500, 4000, 4500, 5000, 5500, 6000, 6,500, 7,000, 7,500, 8,000, 8,500, 9,000, 9,500 or 10,000 or more base pairs to about 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 20,500, 21,000, 21,500, 22,000, 22,500, 23,000, 23,500, 24,000, 24,500, 25,000, 25,500, 26,000, 26,500, 27,000, 27,500, 28,000, 28,500, 29,000, 29,500, 30,000, 30,500, 31,000, 31,500, 32,000, 32,500, 33,000, 33,500, 34,000, 34,500, 35,000, 35,500, 36,000, 36,500, 37,000, 37,500, 38,000, 38,500, 39,000, 39,500, 40,000, 40,500, 41,000, 41,500, 42,000, 42,500, 43,000, 43,500, 44,000, 44,500, 45,000, 45,500, 46,000, 46,500, 47,000, 47,500, 48,000, 48,500, 49,000, 49,500 or 50,000 or more base pairs. The structural variant can be on the same arm of the chromosome as the CD274 gene and/or the CD273 gene (i.e., the “p” arm of chromosome 9), or it can be on the “q” arm of chromosome 9. In aspects, the distance is measured from the 3′ end of the CD274 gene. In certain aspects, the distance, as measured from the 5′ end or the 3′ end of the CD274 gene, is between about 4,400 base pairs to about 25,000 base pairs. In certain aspects of any of the methods provided herein, the adjacent location of the structural variant is at a distance of about 4,447 base pairs from the CD274 gene, where the distance is measured from the 3′ end of the CD274 gene. In aspects, the adjacent location of the structural variant is at a distance of between a closest distance of about 4,447 base pairs, where the distance is measured from the 3′ end of the CD274 gene, to a farthest distance of about 24,459 base pairs from the CD274 gene, where the distance is measured from the 5′ end of the CD274 gene. In aspects, the distance is a linear distance. In certain aspects of any of the methods provided herein, the adjacent location of the structural variant and/or a breakpoint of the structural variant, is at a distance of about 30,000 base pairs to about 35,000 base pairs from the CD273 gene, where the distance is measured from the 5′ end of the CD273 gene. In aspects, the adjacent location of the structural variant is at a distance of about 30,000 base pairs, 31,000 base pairs, 32,000 base pairs, 33,000 base pairs, 34,000 base pairs or 35,000 base pairs from the CD273 gene, where the distance is measured from the 5′ end of the CD273 gene. In aspects, the adjacent location of the structural variant and/or a breakpoint of the structural variant is at a closest distance of between about 33,000 base pairs to about 35,000 base pairs from the CD273 gene. In aspects, the adjacent location of the structural variant and/or a breakpoint of the structural variant is at a closest distance of about 34,531 base pairs from the CD273 gene. In certain aspects, the distance is a linear distance.

In aspects, the structural variant is a translocation. In certain aspects, the structural variant includes an ectopic portion of genomic DNA from positions 18,377,000 to 18,386,000 on chromosome 9. In aspects, the structural variant comprises an ectopic portion of genomic DNA from positions 18,381,001 to 18,382,000 on chromosome 9. In certain aspects, the ectopic portion is on the p arm of chromosome 9. In aspects, the translocated ectopic portion of genomic DNA is between positions 5,475,001 to 5,476,000 on chromosome 9. In aspects, the positions referred to herein are in reference to an HG38 human reference genome.

### Structural Variants

Provided herein are methods for treating a subject identified as having a structural variant adjacent to the CD274 gene, having a structural variant adjacent to the CD273 gene, or having a structural variant adjacent to the CD274 gene and having a structural variant adjacent to the CD273 gene. A structural variant may be referred to as a structural variation and/or a chromosomal rearrangement. A structural variant may comprise one or more of a translocation, inversion, insertion, deletion, and duplication. In some embodiments, a structural variant comprises a microduplication and/or a microdeletion. In some embodiments, a structural variant comprises a fusion (e.g., a gene fusion where a portion of a first gene is inserted into a portion of a second gene). Any type of structural variant, including, but not limited to, a translocation, insertion, inversion, deletion, duplication and the like, as described below, can be of any length and, in some embodiments, is about 1 base or base pair (bp) to about 250 megabases (Mb) in length. In some embodiments, a structural variation is about 1 base or base pair (bp) to about 50,000 kilobases (kb) in length (e.g., about 10 bp, 50 bp, 100 bp, 500 bp, 1 kb, 5 kb, 10 kb, 50 kb, 100 kb, 500 kb, 1000 kb, 5000 kb or 10,000 kb in length). A structural variant may be intra-chromosomal (rearrangement of genomic material within a chromosome) or inter-chromosomal (rearrangement of genomic material between two or more chromosomes).

A structural variant may comprise a translocation. A translocation is a genetic event that results in a rearrangement of chromosomal material. Translocations may include reciprocal translocations and Robertsonian translocations. A reciprocal translocation is a chromosome abnormality caused by exchange of parts between non-homologous chromosomes-two detached fragments of two different chromosomes are switched. A Robertsonian translocation occurs when two non-homologous chromosomes become attached, meaning that given two healthy pairs of chromosomes, one of each pair sticks and blends together homogeneously. A gene fusion may be created when a translocation joins two genes that are normally separate.

Translocations may be balanced (i.e., in an even exchange of material with no genetic information extra or missing, sometimes with full functionality) or unbalanced (i.e., where the exchange of chromosome material is unequal resulting in extra or missing genes or fragments thereof).

A structural variant may comprise an inversion. An inversion is a chromosome rearrangement in which a segment of a chromosome is reversed end-to-end. An inversion may occur when a single chromosome undergoes breakage and rearrangement within itself. Inversions may be of two types: paracentric and pericentric. Paracentric inversions do not include the centromere, and both breaks occur in one arm of the chromosome. Pericentric inversions include the centromere, and there is a break point in each arm.

A structural variant may comprise an insertion. An insertion may be the addition of one or more nucleotide base pairs into a nucleic acid sequence. An insertion may be a microinsertion, e.g., generally a submicroscopic insertion of any length ranging from 1 base to about 10 megabases, such as from about 1 megabase to about 3 megabases. In certain embodiments, an insertion comprises the addition of a segment of a chromosome into a genome, chromosome, or segment thereof. In certain embodiments an insertion comprises the addition of an allele, a gene, an intron, an exon, any non-coding region, any coding region, segment thereof or combination thereof into a genome or segment thereof. In certain embodiments an insertion comprises the addition (e.g., insertion) of nucleic acid of unknown origin into a genome, chromosome, or segment thereof. In certain embodiments an insertion comprises the addition (e.g., insertion) of a single base.

A structural variant may comprise a deletion. In certain embodiments, a deletion is a genetic aberration in which a part of a chromosome or a sequence of DNA is missing. A deletion can, in certain embodiments, result in the loss of genetic material. In embodiments, a deletion can be translocated to another portion of the genome (balanced translocation or unbalanced translocation), such as on the same chromosome (same arm of the chromosome or other arm of the chromosome) or on a different chromosome. Any number of nucleotides can be deleted. A deletion can comprise the deletion of one or more entire chromosomes, a segment of a chromosome, an allele, a gene, an intron, an exon, any non-coding region, any coding region, a segment thereof or combination thereof. A deletion can include a microdeletion, e.g., generally a submicroscopic deletion of any length ranging from 1 base to about 10 megabases, such as from about 1 megabase to about 3 megabases. A deletion can include the deletion of a single base.

A structural variant may comprise a duplication. In certain embodiments, a duplication is a genetic aberration in which a part of a chromosome or a sequence of DNA is copied and inserted back into the genome. In certain embodiments, a duplication is any duplication of a region of DNA. In some embodiments, a duplication is a nucleic acid sequence that is repeated, often in tandem, within a genome or chromosome. In some embodiments a duplication can comprise a copy of one or more entire chromosomes, a segment of a chromosome, an allele, a gene, an intron, an exon, any non-coding region, any coding region, segment thereof or combination thereof. A duplication can include a microduplication, e.g., generally a submicroscopic duplication of any length ranging from 1 base to about 10 megabases, such as from about 1 megabase to about 3 megabases. A duplication sometimes comprises one or more copies of a duplicated nucleic acid. A duplication may be characterized as a genetic region repeated one or more times (e.g., repeated 1, 2, 3, 4, 5, 6, 7, 8, 9 or 10 times). Duplications can range from small regions (thousands of base pairs) to whole chromosomes in some instances. Duplications may occur as the result of an error in homologous recombination or due to a retrotransposon event.

A structural variant may be intra-chromosomal (rearrangement of genomic material within a chromosome) or inter-chromosomal (rearrangement of genomic material between two or more chromosomes). A structural variant may include a plurality of chromosomal rearrangements (e.g., translocations, inversions, insertions, deletions, duplications). For example, a structural variant may include a plurality of intra-chromosomal rearrangements. In certain instances, a structural variant may include a plurality of inter-chromosomal rearrangements. In certain instances, a structural variant may include a plurality of intra-chromosomal rearrangements and inter-chromosomal rearrangements.

### Breakpoints and Donor/Receiver Sites

A structural variant may be defined according to one or more breakpoints. A breakpoint generally refers to a genomic position (i.e., genomic coordinate) where a structural variant occurs (e.g., translocation, inversion, insertion, deletion, or duplication). A breakpoint may refer to a genomic position where an ectopic portion of genomic material is inserted (e.g., a recipient site for an insertion or a translocation). A breakpoint may refer to a genomic position where a portion of genomic material is deleted (e.g., a donor site for an insertion or a translocation). A breakpoint may refer to a pair of genomic positions (i.e., genomic coordinates) that have become flanking (i.e., adjacent) to one another as a result of a structural variant (e.g., translocation, inversion, insertion, deletion, or duplication). A breakpoint may be defined in terms of a position or positions in a reference genome. A breakpoint may be defined in terms of a position or positions in a human reference genome (e.g., HG38 human reference genome). Generally, genomic positions discussed herein are in reference to an HG38 human reference genome, and corresponding and/or equivalent positions in any other human reference genome are contemplated herein.

A breakpoint may be defined in terms mapping to a position or positions in a reference genome. A breakpoint may be defined in terms of mapping to a position or positions in a human reference genome (e.g., HG38 human reference genome). A breakpoint may map to a position in a reference genome when a nucleic acid sequence located upstream, downstream, or spanning the breakpoint aligns with a corresponding sequence in a reference genome. Any suitable mapping method (e.g., process, algorithm, program, software, module, the like or combination thereof) can be used and certain aspects of mapping processes are described hereafter.

Mapping a nucleic acid sequence may comprise mapping one or more nucleic acid sequence reads (e.g., sequence information from a fragment whose physical genomic position is unknown), which can be performed in a number of ways, and often comprises alignment of the obtained sequence reads with a matching sequence in a reference genome. In such alignments, sequence reads generally are aligned to a reference sequence and those that align are designated as being “mapped”, “a mapped sequence read” or “a mapped read”.

The terms “aligned”, “alignment”, or “aligning” generally refer to two or more nucleic acid sequences that can be identified as a match (e.g., 100% identity) or partial match. Alignments can be done manually or by a computer (e.g., a software, program, module, or algorithm), non-limiting examples of which include the Efficient Local Alignment of Nucleotide Data (ELAND) computer program distributed as part of the Illumina Genomics Analysis pipeline. Alignment of a sequence read can be a 100% sequence match. In some cases, an alignment is less than a 100% sequence match (e.g., non-perfect match, partial match, partial alignment). In some embodiments an alignment is about a 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76% or 75% match. In some embodiments, an alignment contains a mismatch, such as a base not correctly paired with its canonical Watson-Crick base partner, e.g., A or T incorrectly paired with G or C. In some embodiments, an alignment comprises 1, 2, 3, 4 or 5 mismatches. Two or more sequences can be aligned using either strand. In certain embodiments a nucleic acid sequence is aligned with the reverse complement of another nucleic acid sequence. In certain instances, extra or missing bases within a sequence are expressed as gaps in an alignment and may or may not be factored into a percent identity calculation. For example, a percent identity calculation may include a number of mismatches and gaps or may include a number of mismatches only.

Various computational methods can be used to map and/or align sequence reads to a reference genome. Non-limiting examples of computer algorithms that can be used to align sequences include, without limitation, BLAST, BLITZ, FASTA, BOWTIE 1, BOWTIE 2, ELAND, MAQ, PROBEMATCH, SOAP or SEQMAP, or variations thereof or combinations thereof. In some embodiments, sequence reads can be aligned with reference sequences and/or sequences in a reference genome. In some embodiments, the sequence reads can be found and/or aligned with sequences in nucleic acid databases known in the art including, for example, GenBank, dbEST, dbSTS, EMBL (European Molecular Biology Laboratory) and DDBJ (DNA Databank of Japan). BLAST or similar tools can be used to search the identified sequences against a sequence database.

In some embodiments, a breakpoint of a structural variant maps to a particular location within a range of positions on a particular chromosome. In some embodiments, a breakpoint (e.g., receiving site) of a structural variant (e.g., insertion, translocation) maps to a particular location within a range of positions on a particular chromosome. In some embodiments, a breakpoint of a structural variant maps to a location between positions 5,471,000 to 5,480,000 on chromosome 9. In some embodiments, a breakpoint of a structural variant maps to a location between positions 5,475,001 to 5,476,000 on chromosome 9.

In some embodiments, a breakpoint (e.g., donor site) of a structural variant (e.g., insertion, translocation) maps to a particular location within a range of positions on a particular chromosome. A breakpoint for a donor site may map to a particular location within a range of positions that is different from the location of a receiving site. A breakpoint for a donor site may map to a particular location that is on the same chromosome as a receiving site or may map to a particular location that is on a different chromosome than a receiving site. In some embodiments, a breakpoint of a structural variant maps to a location between positions 18,377,000 to 18,386,000 on chromosome 9. In some embodiments, a breakpoint of a structural variant maps to a location between positions 18,381,001 to 18,382,000 on chromosome 9.

A structural variant may be defined in terms of a receiving site and a donor site. A receiving site may be referred to as a first partner or “partner 1” and a donor site may be referred to as a second partner or “partner 2.” In some embodiments, a structural variant may be defined in terms of comprising an ectopic portion of genomic DNA (i.e., a portion of genomic DNA at a receiving site from a different region of a chromosome or from a different chromosome). The ectopic portion may be referred to as a donor portion. The ectopic portion may comprise genomic DNA from a “donor site” genomic coordinate window provided below, or part thereof. The ectopic portion may comprise genomic DNA from a “donor site” genomic coordinate window provided below, or part thereof, and may further comprise genomic DNA from a region outside of a “donor site” genomic coordinate window provided below.

In some embodiments, a receiving site of a structural variant maps to a location between positions 5,471,000 to 5,480,000 on chromosome 9. In some embodiments, a receiving site of a structural variant maps to a location between positions 5,475,001 to 5,476,000 on chromosome 9. In some embodiments, a donor site of a structural variant maps to a location between positions 18,377,000 to 18,386,000 on chromosome 9. In some embodiments, a donor site of a structural variant maps to a location between positions 18,381,001 to 18,382,000 on chromosome 9. In some embodiments, a receiving site maps to a location adjacent to or within the CD274 gene. In embodiments, a receiving site maps to a location adjacent to or within the CD273 gene.

In embodiments, a structural variant, or a breakpoint of a structural variant, maps to a location adjacent to or within the CD274 gene. In embodiments, a structural variant, or a breakpoint of a structural variant, maps to a location adjacent to or within the CD273 gene.

In some embodiments, a structural variant may comprise an ectopic portion of genomic DNA (i.e., a portion of genomic DNA at a receiving site from a different region of a chromosome or from a different chromosome). The ectopic portion may be referred to as a donor portion. If the ectopic portion (donor portion) is from the same chromosome as the structural variant, the ectopic portion may be from a location outside of the position ranges provided above for certain structural variants. The ectopic portion may comprise genomic DNA from a genomic coordinate window provided herein, or part thereof. The ectopic portion may comprise genomic DNA from a genomic coordinate window provided herein, or part thereof, and may further comprise genomic DNA from a region outside of a genomic coordinate window provided herein.

In some embodiments, an ectopic portion of genomic DNA is characterized by its location (e.g., observed location for a given sample or samples) at a receiving site (e.g., at a structural variant site). In some embodiments, an ectopic portion is characterized by its location (e.g., observed location for a given sample samples) relative to a coding region of a gene and/or oncogene. A coding region of a gene and/or oncogene generally refers to a part of the gene and/or oncogene that is transcribed and translated into protein (i.e., the sum total of its exons). In some embodiments, an ectopic portion is within a coding region of a gene and/or oncogene. In some embodiments, an ectopic portion is not within a coding region of a gene and/or oncogene. For example, an ectopic portion may be located in an intronic region, an intergenic region, or within another gene. In some embodiments, an ectopic portion is located at a position in proximity to a coding region for a gene and/or oncogene. The term “in proximity” may refer to spatial proximity and/or linear proximity.

Spatial proximity generally refers to 3-dimensional chromatin proximity, which may be assessed according to a method that preserves spatial-proximal relationships, such as a method described herein or any suitable method known in the art. An ectopic portion may be located at a position in spatial proximity to a coding region for a gene and/or oncogene when an ectopic portion and a gene and/or oncogene (or a fragment thereof) are ligated in a proximity ligation assay or are bound by a common solid phase in a solid substrate-mediated proximity capture (SSPC) assay, for example.

Linear proximity generally refers to a linear base-pair distance, which may be assessed according to mapped distances in a reference genome, for example. Linear proximity distance may be provided as a distance between a 5′ or 3′ end of an ectopic portion and a 5′ or 3′ end of a gene and/or exon. An ectopic portion may be located at a position in linear proximity to a coding region of a gene and/or oncogene when the ectopic portion is within about 1,000 base pairs, about 2,000 base pairs, about 3,000 base pairs, about 4,000 base pairs, about 5,000 base pairs, about 10,000 base pairs, about 20,000 base pairs, about 30,000 base pairs, about 40,000 base pairs, about 50,000 base pairs, about 60,000 base pairs, about 70,000 base pairs, about 80,000 base pairs, about 90,000 base pairs, about 100,000 base pairs, about 200,000 base pairs, about 300,000 base pairs, about 400,000 base pairs, about 500,000 base pairs, about 600,000 base pairs, about 700,000 base pairs, about 800,000 base pairs, about 900,000 base pairs, or about 1,000,000 base pairs of a coding region of a gene and/or oncogene.

In some embodiments, a structural variant comprises an ectopic portion of genomic DNA from chromosome 9 (donor site). In some embodiments, an ectopic portion is located at a position in chromosome 9 (receiver site) in proximity to a coding region for PD-L1. In some embodiments, an ectopic portion is located at a position in chromosome 9 (receiver site) in proximity to a coding region for PD-L2. In some embodiments, an ectopic portion is located at a position in chromosome 9 in spatial proximity to a coding region for PD-L1. In some embodiments, an ectopic portion is located at a position in chromosome 9 in spatial proximity to a coding region for PD-L2. In some embodiments, an ectopic portion is located at a position in chromosome 9 in linear proximity to a coding region for PD-L1. In some embodiments, an ectopic portion is located at a position in chromosome 9 in linear proximity to a coding region for PD-L2. In some embodiments, an ectopic portion is located at a position in chromosome 9 within about 2,000 base pairs to about 7,000 base pairs, about 3,000 base pairs to about 6,000 base pairs, or about 4,000 base pairs to about 5,000 base pairs to a coding region for PD-L1 (e.g., the 3′ end of the PD-L1 gene). In some embodiments, an ectopic portion is located at a position in chromosome 9 within about 4,447 base pairs to a coding region for PD-L1 (e.g., the 3′ end of the PD-L1 gene). In some embodiments, an ectopic portion is located at a position in chromosome 9 within about 30,000 base pairs to about 40,000 base pairs, about 32,000 base pairs to about 38,000 base pairs, or about 34,000 base pairs to about 35,000 base pairs to a coding region for PD-L2 (e.g., the 5′ end of the PD-L2 gene). In some embodiments, an ectopic portion is located at a position in chromosome 9 within about 34,531 base pairs to a coding region for PD-L2 (e.g., the 5′ end of the PD-L2 gene).

### Genes

A structural variant may be associated with one or more genes and/or genes associated with cancer, referred to herein in general as oncogenes. An oncogene can be any gene that, when altered, is associated with cancer. Alterations may include mutations, structural variants, copy number variations, and the like and combinations thereof. Alterations may be located within an oncogene (i.e., intragenic) or outside of/adjacent to an oncogene (i.e., intergenic, extragenic). In certain instances, alterations may be located within a different gene. Alterations may be located in a portion of genomic DNA that is proximal to a gene and/or an oncogene (e.g., within a certain linear proximity and/or within a certain spatial proximity). Alterations may affect expression of a gene and/or an oncogene (e.g., increased expression, decreased expression, no expression, constitutive expression). Alterations may affect the function of a protein encoded by the gene and/or oncogene (e.g., increased function, decreased function, loss-of-function, gain-of-function, constitutive function, change in function). Non-limiting examples of oncogenes are provided in Table 8. In some embodiments, a structural variant is associated with the CD274 gene. In embodiments, a structural variant is associated with the CD273 gene.

In some embodiments, a structural variant is within a gene (e.g., within an intron and/or exon of a gene (e.g., an oncogene)). In some embodiments, a structural variant is outside of a gene (e.g., within an intergenic region or within a different nearby gene). In some embodiments, a structural variant is adjacent to a gene (e.g., within an intergenic region or within a different nearby gene). Thus, in some embodiments, a breakpoint for a structural variant is not within a gene (e.g., an oncogene). In certain instances, a structural variant (e.g., an intergenic structural variant) may be defined in terms of linear distance to a gene (e.g., an oncogene). Linear distance may be measured from the 5′ end of a gene and/or a 3′ end of a gene. In some embodiments, a breakpoint for a structural variant may be located at least about 1 kb to about 500 kb from the 5′ end or 3′ end of a gene. For example, a breakpoint for a structural variant may be located at least about 1 kb, 2 kb, 3 kb, 4 kb, 5 kb, 10 kb, 20 kb, 30 kb, 40 kb, 50 kb, 60 kb, 70 kb, 80 kb, 90 kb, 100 kb, 200 kb, 300 kb, 400 kb, or 500 kb from the 5′ end or 3′ end of a gene.

In some embodiments, a breakpoint for a structural variant is located at least about 4,000 base pairs from an oncogene, where the oncogene is CD274, and where the distance is measured from the 3′ end of CD274. In some embodiments, a breakpoint for a structural variant is located at a genomic position that is between about 4,000 to 6,000 base pairs from an oncogene, where the oncogene is CD274, and where the distance is measured from the 3′ end of CD274. In some embodiments, a breakpoint for a structural variant is located at least about 4,447 base pairs from an oncogene, where the oncogene is CD274, and where the distance is measured from the 3′ end of CD274. In some embodiments, a breakpoint for a structural variant is located at a genomic position that is between about 4,447 to 5,447 base pairs from an oncogene, where the oncogene is CD274, and where the distance is measured from the 3′ end of CD274.

In some embodiments, a structural variant and/or a breakpoint for a structural variant is located at least about 34,000 base pairs from an oncogene, where the oncogene is CD273, and where the distance is measured from the 5′ end of CD273. In some embodiments, a structural variant and/or a breakpoint for a structural variant is located at a genomic position that is between about 34,000 to about 36,000 base pairs from an oncogene, where the oncogene is CD273, and where the distance is measured from the 5′ end of CD273. In some embodiments, a structural variant and/or a breakpoint for a structural variant is located at least about 34,531 base pairs from an oncogene, where the oncogene is CD273, and where the distance is measured from the 5′ end of CD273. In some embodiments, a structural variant and/or a breakpoint for a structural variant is located at a genomic position that is between about 34,531 to 35,531 base pairs from an oncogene, where the oncogene is CD273, and where the distance is measured from the 5′ end of CD273.

### Nucleic Acid

In aspects of the methods provided herein, the subject having cancer that is selected and/or treated according to the methods provided herein is predetermined as having a structural variant adjacent to the CD274 gene and/or the CD273 gene. In certain aspects, the subject having cancer is selected or screened for treatment according to the methods provided herein by analyzing nucleic acid of a sample obtained from the subject for the presence or absence of a structural variant adjacent to the CD274 gene and/or the CD273 gene. Provided herein are methods and compositions for processing and/or analyzing nucleic acid. The terms nucleic acid(s), nucleic acid molecule(s), nucleic acid fragment(s), target nucleic acid(s), nucleic acid template(s), template nucleic acid(s), nucleic acid target(s), target nucleic acid(s), polynucleotide(s), polynucleotide fragment(s), target polynucleotide(s), polynucleotide target(s), and the like may be used interchangeably throughout the disclosure. The terms refer to nucleic acids of any composition from, such as DNA (e.g., complementary DNA (cDNA; synthesized from any RNA or DNA of interest), genomic DNA (gDNA), genomic DNA fragments, mitochondrial DNA (mtDNA), recombinant DNA (e.g., plasmid DNA), and the like), RNA (e.g., message RNA (mRNA), small interfering RNA (siRNA), ribosomal RNA (rRNA), transfer RNA (tRNA), microRNA, transacting small interfering RNA (ta-siRNA), natural small interfering RNA (nat-siRNA), small nucleolar RNA (snoRNA), small nuclear RNA (snRNA), long non-coding RNA (lncRNA), non-coding RNA (ncRNA), transfer-messenger RNA (tmRNA), precursor messenger RNA (pre-mRNA), small Cajal body-specific RNA (scaRNA), piwi-interacting RNA (piRNA), endoribonuclease-prepared siRNA (esiRNA), small temporal RNA (stRNA), signal recognition RNA, telomere RNA, RNA highly expressed by a fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. A nucleic acid may be, or may be from, a plasmid, phage, virus, bacterium, autonomously replicating sequence (ARS), mitochondria, centromere, artificial chromosome, chromosome, or other nucleic acid able to replicate or be replicated in vitro or in a host cell, a cell, a cell nucleus or cytoplasm of a cell in certain embodiments. A template nucleic acid in some embodiments can be from a single chromosome (e.g., a nucleic acid sample may be from one chromosome of a sample obtained from a diploid organism). Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense,” “plus” strand or “minus” strand, “forward” reading frame or “reverse” reading frame) and double-stranded polynucleotides. The term “gene” refers to a section of DNA involved in producing a polypeptide chain; and generally includes regions preceding and following the coding region (leader and trailer) involved in the transcription/translation of the gene product and the regulation of the transcription/translation, as well as intervening sequences (introns) between individual coding regions (exons). A nucleotide or base generally refers to the purine and pyrimidine molecular units of nucleic acid (e.g., adenine (A), thymine (T), guanine (G), and cytosine (C)). For RNA, the base thymine is replaced with uracil (U). Nucleic acid length or size may be expressed as a number of bases.

Target nucleic acids may be any nucleic acids of interest. Nucleic acids may be polymers of any length composed of deoxyribonucleotides (i.e., DNA bases), ribonucleotides (i.e., RNA bases), or combinations thereof, e.g., 10 bases or longer, 20 bases or longer, 50 bases or longer, 100 bases or longer, 200 bases or longer, 300 bases or longer, 400 bases or longer, 500 bases or longer, 1000 bases or longer, 2000 bases or longer, 3000 bases or longer, 4000 bases or longer, 5000 bases or longer. In certain aspects, nucleic acids are polymers composed of deoxyribonucleotides (i.e., DNA bases), ribonucleotides (i.e., RNA bases), or combinations thereof, e.g., 10 bases or less, 20 bases or less, 50 bases or less, 100 bases or less, 200 bases or less, 300 bases or less, 400 bases or less, 500 bases or less, 1000 bases or less, 2000 bases or less, 3000 bases or less, 4000 bases or less, or 5000 bases or less.

Nucleic acid may be single-stranded or double-stranded. Single-stranded DNA (ssDNA), for example, can be generated by denaturing double-stranded DNA by heating or by treatment with alkali, for example. Accordingly, in some embodiments, ssDNA is derived from double-stranded DNA (dsDNA).

Nucleic acid (e.g., genomic DNA, nucleic acid targets, oligonucleotides, probes, primers) may be described herein as being complementary to another nucleic acid, having a complementarity region, being capable of hybridizing to another nucleic acid, or having a hybridization region. The terms “complementary” or “complementarity” or “hybridization” generally refer to a nucleotide sequence that base-pairs by non-covalent bonds to a region of a nucleic acid. In the canonical Watson-Crick base pairing, adenine (A) forms a base pair with thymine (T), and guanine (G) pairs with cytosine (C) in DNA. In RNA, thymine (T) is replaced by uracil (U). As such, A is complementary to T and G is complementary to C. In RNA, A is complementary to U and vice versa. In a DNA-RNA duplex, A (in a DNA strand) is complementary to U (in an RNA strand). Typically, “complementary” or “complementarity” or “capable of hybridizing” refer to a nucleotide sequence that is at least partially complementary. These terms may also encompass duplexes that are fully complementary such that every nucleotide in one strand is complementary or hybridizes to every nucleotide in the other strand in corresponding positions. In certain instances, a nucleotide sequence may be partially complementary to a target, in which not all nucleotides are complementary to every nucleotide in the target nucleic acid in all the corresponding positions.

The percent identity of two nucleotide sequences can be determined by aligning the sequences for optimal comparison purposes, e.g., when the total number of positions is different between the two nucleotide sequences, gaps may be introduced in the sequence of one or both sequences for optimal alignment. The nucleotides at corresponding positions are then compared, and the percent identity between the two sequences can be determined as a function of the number of identical positions shared by the sequences (e.g., % identity=# of identical positions/total # of positions×100). When a position in one sequence is occupied by the same nucleotide as the corresponding position in the other sequence, then the molecules are identical at that position. In certain instances, extra or missing bases within a sequence are expressed as gaps in an alignment and may or may not be factored into a percent identity calculation. For example, a percent identity calculation may include a number of mismatches and gaps or may include a number of mismatches only.

As used herein, the phrase “hybridizing” or grammatical variations thereof, refers to binding of a first nucleic acid molecule to a second nucleic acid molecule under low, medium or high stringency conditions, or under nucleic acid synthesis conditions. Hybridizing can include instances where a first nucleic acid molecule binds to a second nucleic acid molecule, where the first and second nucleic acid molecules are complementary. As used herein, “specifically hybridizes” refers to preferential hybridization under nucleic acid synthesis conditions of a primer, oligonucleotide, or probe, to a nucleic acid molecule having a sequence complementary to the primer, oligonucleotide, or probe compared to hybridization to a nucleic acid molecule not having a complementary sequence. For example, specific hybridization includes the hybridization of a primer, oligonucleotide, or probe to a target nucleic acid sequence that is complementary to the primer, oligonucleotide, or probe.

Primer, oligonucleotide, or probe sequences and length can affect hybridization to target nucleic acid sequences. Depending on the degree of mismatch between the primer, oligonucleotide, or probe and target nucleic acid, low, medium or high stringency conditions may be used to effect primer/target, oligonucleotide/target, or probe/target annealing. As used herein, the term “stringent conditions” refers to conditions for hybridization and washing. Methods for hybridization reaction temperature condition optimization are known, and can be found, e.g., in Current Protocols in Molecular Biology, John Wiley & Sons, N.Y., 6.3.1-6.3.6 (1989), as described further below.

In some embodiments, target nucleic acids comprise degraded DNA. Degraded DNA may be referred to as low-quality DNA or highly degraded DNA. Degraded DNA may be highly fragmented, and may include damage such as base analogs and abasic sites subject to miscoding lesions and/or intermolecular crosslinking. For example, sequencing errors resulting from deamination of cytosine residues may be present in certain sequences obtained from degraded DNA (e.g., miscoding of C to T and G to A).

Nucleic acid may be derived from one or more sources (e.g., a biological sample described herein) by methods known in the art. Any suitable method can be used for isolating, extracting and/or purifying DNA from a biological sample (e.g., from blood or a blood product, tissue, tumor), non-limiting examples of which include methods of DNA preparation, various commercially available reagents or kits, such as DNeasy®, RNeasy®, QIAprep®, QIAquick®, and QIAamp® (e.g., QIAamp® Circulating Nucleic Acid Kit, QiaAmp® DNA Mini Kit or QiaAmp® DNA Blood Mini Kit) nucleic acid isolation/purification kits by Qiagen, Inc. (Germantown, Md); GenomicPrep™ Blood DNA Isolation Kit (Promega, Madison, Wis.); GFX™ Genomic Blood DNA Purification Kit (Amersham, Piscataway, N.J.); DNAzol®, ChargeSwitch®, Purelink®, GeneCatcher® nucleic acid isolation/purification kits by Life Technologies, Inc. (Carlsbad, CA); NucleoMag®, NucleoSpin®, and NucleoBond® nucleic acid isolation/purification kits by Clontech Laboratories, Inc. (Mountain View, CA); the like or combinations thereof. In certain aspects, nucleic acid is isolated from a fixed biological sample, e.g., formalin-fixed, paraffin-embedded (FFPE) tissue. Genomic DNA from FFPE tissue may be isolated using commercially available kits—such as the AllPrep® DNA/RNA FFPE kit by Qiagen, Inc. (Germantown, Md), the RecoverAll® Total Nucleic Acid Isolation kit for FFPE by Life Technologies, Inc. (Carlsbad, CA), and the NucleoSpin® FFPE kits by Clontech Laboratories, Inc. (Mountain View, CA).

In some embodiments, nucleic acid is extracted from cells using a cell lysis procedure. Cell lysis procedures and reagents are known in the art and may generally be performed by chemical (e.g., detergent, hypotonic solutions, enzymatic procedures, and the like, or combination thereof), physical (e.g., French press, sonication, and the like), or electrolytic lysis methods. Any suitable lysis procedure can be utilized. For example, chemical methods generally employ lysing agents to disrupt cells and extract the nucleic acids from the cells, followed by treatment with chaotropic salts. Physical methods such as freeze/thaw followed by grinding, the use of cell presses and the like also are useful. In some instances, a high salt and/or an alkaline lysis procedure may be utilized. In some instances, a lysis procedure may include a lysis step with EDTA/Proteinase K, a binding buffer step with high amount of salts (e.g., guanidinium chloride (GuHCl), sodium acetate) and isopropanol, and binding DNA in this solution to silica-based column.

Nucleic acids can include extracellular nucleic acid in certain embodiments. The term “extracellular nucleic acid” as used herein can refer to nucleic acid isolated from a source having substantially no cells and also is referred to as “cell-free” nucleic acid (cell-free DNA, cell-free RNA, or both), “circulating cell-free nucleic acid” (e.g., CCF fragments, ccfDNA) and/or “cell-free circulating nucleic acid.” Extracellular nucleic acid can be present in and obtained from blood (e.g., from the blood of a human subject). Extracellular nucleic acid often includes no detectable cells and may contain cellular elements or cellular remnants. Non-limiting examples of acellular sources for extracellular nucleic acid are blood, blood plasma, blood serum and urine. In certain aspects, cell-free nucleic acid is obtained from a body fluid sample chosen from whole blood, blood plasma, blood serum, amniotic fluid, saliva, urine, pleural effusion, bronchial lavage, bronchial aspirates, breast milk, colostrum, tears, seminal fluid, peritoneal fluid, pleural effusion, and stool. As used herein, the term “obtain cell-free circulating sample nucleic acid” includes obtaining a sample directly (e.g., collecting a sample, e.g., a test sample) or obtaining a sample from another who has collected a sample. Extracellular nucleic acid may be a product of cellular secretion and/or nucleic acid release (e.g., DNA release). Extracellular nucleic acid may be a product of any form of cell death, for example. In some instances, extracellular nucleic acid is a product of any form of type I or type II cell death, including mitotic, oncotic, toxic. ischemic, and the like and combinations thereof. Without being limited by theory, extracellular nucleic acid may be a product of cell apoptosis and cell breakdown, which provides basis for extracellular nucleic acid often having a series of lengths across a spectrum (e.g., a “ladder”). In some instances, extracellular nucleic acid is a product of cell necrosis, necropoptosis, oncosis, entosis, pyrotosis, and the like and combinations thereof. In some embodiments, sample nucleic acid from a test subject is circulating cell-free nucleic acid. In some embodiments, circulating cell free nucleic acid is from blood plasma or blood serum from a test subject. In some aspects, cell-free nucleic acid is degraded. In certain aspects, cell-free nucleic acid comprises circulating cancer nucleic acid (e.g., cancer DNA). In certain aspects, cell-free nucleic acid comprises circulating tumor nucleic acid (e.g., tumor DNA).

Extracellular nucleic acid can include different nucleic acid species, and therefore is referred to herein as “heterogeneous” in certain embodiments. For example, blood serum or plasma from a person having a tumor or cancer can include nucleic acid from tumor cells or cancer cells (e.g., neoplasia) and nucleic acid from non-tumor cells or non-cancer cells. In some instances, cancer nucleic acid and/or tumor nucleic acid sometimes is about 5% to about 50% of the overall nucleic acid (e.g., about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49% of the total nucleic acid is cancer, or tumor nucleic acid).

Nucleic acid may be provided for conducting methods described herein with or without processing of the sample(s) containing the nucleic acid. In some embodiments, nucleic acid is provided for conducting methods described herein after processing of the sample(s) containing the nucleic acid. For example, a nucleic acid can be extracted, isolated, purified, partially purified or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” as used herein can refer to a nucleic acid removed from a subject (e.g., a human subject). An isolated nucleic acid can be provided with fewer non-nucleic acid components (e.g., protein, lipid) than the amount of components present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%, 97%, 98%, 99% or greater than 99% free of non-nucleic acid components. The term “purified” as used herein can refer to a nucleic acid provided that contains fewer non-nucleic acid components (e.g., protein, lipid, carbohydrate) than the amount of non-nucleic acid components present prior to subjecting the nucleic acid to a purification procedure. A composition comprising purified nucleic acid may be about 80%, 81%, 82%, 83%, 84%, 85%, 86%, 87%, 88%, 89%, 90%, 91%, 92%, 93%, 94%, 95%, 96%, 97%, 98%, 99% or greater than 99% free of other non-nucleic acid components. The term “purified” as used herein can refer to a nucleic acid provided that contains fewer nucleic acid species than in the sample source from which the nucleic acid is derived. A composition comprising purified nucleic acid may be about 90%, 91%, 92%, 93%, 94%, 95%, 96%, 97%, 98%, 99% or greater than 99% free of other nucleic acid species. In certain examples, small fragments of nucleic acid (e.g., 30 to 500 bp fragments) can be purified, or partially purified, from a mixture comprising nucleic acid fragments of different lengths. In certain examples, nucleosomes comprising smaller fragments of nucleic acid can be purified from a mixture of larger nucleosome complexes comprising larger fragments of nucleic acid. In certain examples, larger nucleosome complexes comprising larger fragments of nucleic acid can be purified from nucleosomes comprising smaller fragments of nucleic acid. In certain examples, cancer cell nucleic acid can be purified from a mixture comprising cancer cell and non-cancer cell nucleic acid. In certain examples, nucleosomes comprising small fragments of cancer cell nucleic acid can be purified from a mixture of larger nucleosome complexes comprising larger fragments of non-cancer nucleic acid. In some embodiments, nucleic acid is provided for conducting methods described herein without prior processing of the sample(s) containing the nucleic acid. For example, nucleic acid may be analyzed directly from a sample without prior extraction, purification, partial purification, and/or amplification.

### Nucleic Acid Analysis

A method herein may comprise one or more nucleic acid analyses. For example, nucleic acid obtained from a sample from a subject may be analyzed for the presence or absence of a structural variant. Any suitable process for detecting a structural variant in a nucleic acid sample may be used. Non-limiting examples of processes for analyzing nucleic acid include amplification (e.g., polymerase chain reaction (PCR)), targeted sequencing, microarray, and fluorescence in situ hybridization (FISH), methods that preserve spatial-proximal relationships and/or spatial-proximal contiguity information, and methods that generate proximity ligated nucleic acid molecules.

In some embodiments, a nucleic acid analysis comprises nucleic acid amplification. For example, nucleic acids may be amplified under amplification conditions. The term “amplified” or “amplification” or “amplification conditions” generally refer to subjecting a target nucleic acid in a sample to a process that linearly or exponentially generates amplicon nucleic acids having the same or substantially the same nucleotide sequence as the target nucleic acid, or part thereof. In certain embodiments, the term “amplified” or “amplification” or “amplification conditions” refers to a method that comprises a polymerase chain reaction (PCR). Detecting a structural variant (SV) described herein using amplification (e.g., PCR) may include use of primers designed to hybridize to a region upstream (e.g., 5′) of one or more SV breakpoints, hybridize to a region downstream (e.g., 3′) of one or more SV breakpoints, hybridize to a region adjacent to one or more SV breakpoints, and/or hybridize to a region spanning one or more SV breakpoints. Examples of PCR primers useful for identifying a structural variant are provided herein.

In some embodiments, a nucleic acid analysis comprises fluorescence in situ hybridization (FISH). Fluorescence in situ hybridization (FISH) is a technique that uses fluorescent probes that bind to a nucleic acid sequence with a high degree of sequence complementarity. In certain configurations, fluorescence microscopy may be used to observe where the fluorescent probe is bound to a chromosome. Detecting a structural variant (SV) described herein using fluorescence in situ hybridization (FISH) may include use of probes designed to hybridize to a region upstream (e.g., 5′) of one or more SV breakpoints, hybridize to a region downstream (e.g., 3′) of one or more SV breakpoints, hybridize to a region adjacent to one or more SV breakpoints, and/or hybridize to a region spanning one or more SV breakpoints. Examples of probes useful for identifying a structural variant are provided herein.

In some embodiments, a nucleic acid analysis comprises a microarray (e.g., a DNA microarray, DNA chip, biochip). A DNA microarray is a collection of DNA probes attached to a solid surface. Probes can be short sections of a gene or other genomic DNA element that can hybridize to target nucleic acids in a sample (e.g., under high-stringency conditions). Probe-target hybridization is usually detected and quantified by detection of fluorophore-, silver-, or chemiluminescence-labeled targets to determine presence, absence, and/or relative abundance of target nucleic acid sequences in the sample. Detecting a structural variant (SV) described herein using DNA microarrays may include use of array probes designed to hybridize to a region upstream (e.g., 5′) of one or more SV breakpoints, hybridize to a region downstream (e.g., 3′) of one or more SV breakpoints, hybridize to a region adjacent to one or more SV breakpoints, and/or hybridize to a region spanning one or more SV breakpoints. Examples of array probes useful for identifying a structural variant are provided herein.

In some embodiments, a nucleic acid analysis comprises sequencing (e.g., genome-wide sequencing, targeted sequencing). Nucleic acid can be sequenced using any suitable sequencing platform, non-limiting examples of which include Maxim & Gilbert, chain-termination methods, sequencing by synthesis, sequencing by ligation, sequencing by mass spectrometry, microscopy-based techniques, the like or combinations thereof. In some embodiments, a first-generation technology, such as, for example, Sanger sequencing methods including automated Sanger sequencing methods, including microfluidic Sanger sequencing, can be used in a method provided herein. In some embodiments, sequencing technologies that include the use of nucleic acid imaging technologies (e.g., transmission electron microscopy (TEM) and atomic force microscopy (AFM)), can be used. In embodiments, a high-throughput sequencing method can be used. High-throughput sequencing methods generally involve clonally amplified DNA templates or single DNA molecules that are sequenced in a massively parallel fashion, sometimes within a flow cell. Next generation (e.g., 2nd and 3rd generation) sequencing techniques capable of sequencing DNA in a massively parallel fashion can be used for methods described herein and are collectively referred to herein as “massively parallel sequencing” (MPS). In embodiments, MPS sequencing methods utilize a targeted approach, where specific chromosomes, genes or regions of interest are sequenced.

Non-limiting examples of sequencing platforms include a sequencing platform provided by Illumina® (e.g., HiSeq™, HiSeq™ 2000, MiSeq™, Genome Analyzer™, and Genome Analyzer™ ∥ sequencing systems); Oxford Nanopore™ Technologies (e.g., MinION sequencing system), Ion Torrent™ (e.g., Ion PGM™ and/or Ion Proton™ sequencing systems); Pacific Biosciences (e.g., PACBIO RS II sequencing system); Life Technologies™ (e.g., SOLID sequencing system); Roche (e.g., 454 GS FLX+ and/or GS Junior sequencing systems); Helicos True Single Molecule Sequencing; Ion semiconductor-based sequencing (e.g., as developed by Life Technologies), WildFire, 5500, 5500×l W and/or 5500×l W Genetic Analyzer based technologies (e.g., as developed and sold by Life Technologies, U.S. Patent Application Publication No. 2013/0012399); Polony sequencing, Pyrosequencing, Massively Parallel Signature Sequencing (MPSS), RNA polymerase (RNAP) sequencing, LaserGen systems and methods, Nanopore-based platforms, chemical-sensitive field effect transistor (CHEMFET) array, electron microscopy-based sequencing (e.g., as developed by ZS Genetics, Halcyon Molecular), nanoball sequencing; or any other suitable sequencing platform. Other sequencing methods that can be used to conduct methods herein include digital PCR, sequencing by hybridization, nanopore sequencing, chromosome-specific sequencing (e.g., using DANSR (digital analysis of selected regions) technology).

In certain embodiments, the sequencing process is a highly multiplexed sequencing process. In certain instances, a full or substantially full sequence is obtained and sometimes a partial sequence is obtained.

For targeted sequencing, a target nucleic acid may be amplified (e.g., by PCR with primers specific to the target), enriched using a probe-based approach, where one or more probes hybridize to a target nucleic acid prior to sequencing, or enriched using Cas9-mediated approaches, such as Cas9-guided adapter ligation, as described in Gilpatrick, T. et al., Targeted nanopore sequencing with Cas9-guided adapter ligation, Nature Biotechnology, volume 38, pages 433-438 (2020). Nucleic acid may be sequenced using any suitable sequencing platform including a Sanger sequencing platform, a high throughput or massively parallel sequencing (next generation sequencing (NGS)) platform, or the like, such as, for example, a sequencing platform provided by Illumina® (e.g., HiSeq™, MiSeq™ and/or Genome Analyzer™ sequencing systems); Oxford Nanopore™ Technologies (e.g., MinION sequencing system), Ion Torrent™ (e.g., Ion PGM™ and/or Ion Proton™ sequencing systems); Pacific Biosciences (e.g., PACBIO RS II sequencing system); Life Technologies™ (e.g., SOLID sequencing system); Roche (e.g., 454 GS FLX+ and/or GS Junior sequencing systems); or any other suitable sequencing platform. In some embodiments, the sequencing process is a highly multiplexed sequencing process. In certain instances, a full or substantially full sequence is obtained and sometimes a partial sequence is obtained. Nucleic acid sequencing generally produces a collection of sequence reads. As used herein, “reads” (e.g., “a read,” “a sequence read”) are short sequences of nucleotides produced by any sequencing process described herein or known in the art. Reads can be generated from one end of nucleic acid fragments (single-end reads), and sometimes are generated from both ends of nucleic acid fragments (e.g., paired-end reads, double-end reads). In some embodiments, a sequencing process generates short sequencing reads or “short reads.” In some embodiments, the nominal, average, mean or absolute length of short reads sometimes is about 10 continuous nucleotides to about 250 or more contiguous nucleotides. In some embodiments, the nominal, average, mean or absolute length of short reads sometimes is about 50 continuous nucleotides to about 150 or more contiguous nucleotides. The length of a sequence read often is associated with the particular sequencing technology utilized. High-throughput methods, for example, provide sequence reads that can vary in size from tens to hundreds of base pairs (bp). Nanopore sequencing, for example, can provide sequence reads that can vary in size from tens to hundreds to thousands of base pairs. In some embodiments, sequence reads are of a mean, median, average or absolute length of about 15 bp to about 900 bp long. In certain embodiments sequence reads are of a mean, median, average or absolute length of about 1000 bp or more. In some embodiments, sequence reads are of a mean, median, average or absolute length of about 100 bp to about 200 bp.

Reads generally are representations of nucleotide sequences in a physical nucleic acid. For example, in a read containing an ATGC depiction of a sequence, “A” represents an adenine nucleotide, “T” represents a thymine nucleotide, “G” represents a guanine nucleotide and “C” represents a cytosine nucleotide, in a physical nucleic acid.

In some embodiments, a nucleic acid analysis comprises a method that preserves spatial-proximal relationships and/or spatial proximal contiguity information (see e.g., International PCT Application Publication No. WO2019/104034; International PCT Application Publication No. WO2020/106776; International PCT Application Publication No. WO2020236851; Kempfer, R., & Pombo, A. (2019). Methods for mapping 3D chromosome architecture. Nature Reviews Genetics. doi: 10.1038/s41576-019-0195-2; and Schmitt, Anthony D.; Hu, Ming; Ren, Bing (2016). Genome-wide mapping and analysis of chromosome architecture. Nature Reviews Molecular Cell Biology. doi: 10.1038/nrm.2016.104; each of which is incorporated by reference in its entirety, to the extent permitted by law). Methods that preserve spatial-proximal relationships and/or spatial proximal contiguity information generally refer to methods that capture and preserve the native spatial conformation exhibited by nucleic acids when associated with proteins as in chromatin and/or as part of a nuclear matrix. Spatial-proximal contiguity information can be preserved by proximity ligation, by solid substrate-mediated proximity capture (SSPC), by compartmentalization with or without a solid substrate or by use of a Tn5 tetramer. Methods that preserve spatial-proximal contiguity information may be based on proximity ligation or may be based on a different principle where special proximity is inferred. Methods based on proximity ligation may include, for example, 3C, 4C, 5C, Hi-C, TCC, GCC, TLA, PLAC-seq, HiChIP, ChIA-PET, Capture-C, Capture-HiC, single-cell HiC, sciHiC, single-cell 3C, single-cell methyl-3C, DNAase HiC, Micro-C, Tiled-C, and Low-C. Methods where special proximity is inferred based on a principle other than proximity ligation may include, for example, SPRITE, scSPRITE, Genome Architecture Mapping (GAM), ChIA-Drop, imaging-based approaches using labeled probes and visualization of DNA, and plus/minus sequencing of an imaged sample (e.g. in situ Genome Sequencing (IGS)). In some embodiments, a nucleic acid analysis comprises generating proximity ligated nucleic acid molecules (e.g., using a method described herein). In some embodiments, a nucleic acid analysis comprises sequencing the proximity ligated nucleic acid molecules, e.g., by a suitable sequencing process known in the art or described herein.

In some embodiments, a nucleic acid analysis comprises a method for preparing nucleic acids from particular types of samples that preserves spatial-proximal relationships and/or spatial-proximal contiguity information in the sequence of the nucleic acids. Nucleic acid molecules that preserve spatial-proximal contiguity information can fragmented and sequenced using short-read sequencing methods (e.g., Illumina, nucleic acid fragments of lengths approximately 500 bp) or intact molecules that preserve spatial-proximal contiguity information can be sequenced using long-read sequencing (e.g., Illumina, Oxford Nanopore, or others, nucleic acid fragments of lengths approximately 30 K bp or greater).

In certain embodiments, a sample can be a fixed sample that is embedded in a material such as paraffin (wax). In some embodiments, a sample can be a formalin fixed sample. In certain embodiments, a sample is formalin-fixed paraffin-embedded (FFPE) sample. In some embodiments, a formalin-fixed paraffin-embedded sample can be a tissue sample or a cell culture sample. In some embodiments, a tissue sample has been excised from a patient and can be diseased or damaged. In some embodiments, a tissue sample is not known to be diseased or damaged. In certain embodiments, a formalin-fixed paraffin-embedded sample can be a formalin-fixed paraffin-embedded section, block, scroll or slide. In certain embodiments, a sample can be a deeply formalin-fixed sample, as described below.

In certain embodiments, a formalin-fixed paraffin-embedded sample is provided on a solid surface and a method of preparing nucleic acid that preserves spatial-proximal contiguity information is performed on the solid surface. In some embodiments, a solid surface is a pathology slide. In some embodiments, additional downstream reactions are also performed on the solid surface.

Those of skill in the art are familiar with methods that can be substituted for steps requiring centrifugation and that achieve a comparable result but are performed on a solid surface.

In some embodiments, methods that preserve spatial-proximal contiguity information comprise methods that generate proximity ligated nucleic acid molecules (e.g., using proximity ligation). A proximity ligation method is one in which natively occurring spatially proximal nucleic acid molecules are captured by ligation to generate ligated products. Proximity ligation methods generally capture spatial-proximal contiguity information in the form of ligation products, whereby a ligation junction is formed between two natively spatially proximal nucleic acids. Once the ligation products are formed, the spatial-proximal contiguity information is detected using next generation sequencing, whereby one or more ligation junctions (either from an entire ligation product or fragment of a ligation product) are sequenced (as described herein). With this sequence information, one is informed that the nucleic acid molecules from a given ligation product (or ligation junction) are natively spatially proximal nucleic acids. In some embodiments, reagents that generate proximity ligated nucleic acid molecules can include a restriction endonuclease, a DNA polymerase, a plurality of nucleotides comprising at least one biotinylated nucleotide, and a ligase. In certain embodiments, two or more restriction endonucleases are used.

Any suitable method for carrying out proximity ligation may be used. For example, a HiC method typically includes the following steps: (1) digestion of chromatin of a solubilized and decompacted FFPE sample with a restriction enzyme (or fragmentation); (2) labelling the digested ends by filling in the 5′-overhangs with biotinylated nucleotides; and (3) ligating the spatially proximal digested ends, thus preserving spatial-proximal contiguity information. Once spatial-proximal contiguity information is preserved, further steps in a HiC method may include: purifying and enriching biotin-labelled ligation junction fragments, preparing a library from the enriched fragments and sequencing the library. Another example of a proximity ligation method may include the following steps: (1) digestion of chromatin of the solubilized and decompacted sample with a restriction enzyme (or fragmentation); (2) blunting the digested or fragmented ends or omission of the blunting procedure; and (3) ligating the spatially proximal ends, thus preserving spatial-proximal contiguity information. Once spatial-proximal contiguity information is preserved, further steps can include: using size selection to purify and enrich ligated fragments, which represent ligation junction fragments, preparing a library from the enriched fragments and sequencing the library. In some embodiments, proximity ligated nucleic acid molecules are generated in situ (i.e., within a nucleus). For methods that include Capture HiC, a further step is included where ligation products containing certain nucleic acid sequences are enriched using one or more capture probes (see, e.g., International Patent Application Publication No. WO 2014/168575). A capture probe generally includes a short sequence of nucleotides or oligonucleotide (e.g., 10-500 bases in length) capable of hybridizing to another nucleotide sequence. In some embodiments, a capture probe includes a label, e.g., a label for selectively purifying specific nucleic acid sequences of interest. Labels are discussed herein and can include, for example, a biotin or digoxigenin label. In some embodiments, capture probes are designed according to a panel of sequences and/or genes of interest (e.g., an oncopanel provided herein).

### Oligonucleotides

Provided herein are oligonucleotides for analyzing structural variants adjacent to the CD274 gene, structural variants adjacent to the CD273 gene, or structural variants adjacent to both the CD274 gene and the CD273 gene, whereby subjects having one or more structural variants are selected for treatment. Oligonucleotides may be artificially synthesized. Accordingly, provided herein in certain embodiments are synthetic oligonucleotides. An oligonucleotide generally refers to a nucleic acid (e.g., DNA, RNA) polymer that is distinct from a target nucleic acid (e.g., a target nucleic acid comprising one or more structural variants described herein), and may be referred to as oligos, probes, and/or primers. Oligonucleotides may be short in length (e.g., less than 50 bp, less than 40 bp, less than 30 bp, less than 20 bp, less than 10 bp). In some embodiments, oligonucleotides are between about 10 to about 500 consecutive nucleotides in length. For example, an oligonucleotide may be about 10, 20, 30, 40, 50, 60, 70, 80, 90, 100, 150, 200, 250, 300, 350, 400, 450, or 500 consecutive nucleotides in length.

Oligonucleotides may be designed to hybridize to a region of a sample nucleic acid that is proximal to, adjacent to, and/or spanning a structural variant described herein, or portion thereof. Oligonucleotides may be designed to hybridize to a region of a sample nucleic acid that comprises a receiving site, a donor site, or a combination of a receiving site and a donor site.

Oligonucleotides may include probes and/or primers useful for detecting presence, absence, or amount of a structural variant in a nucleic acid sample. Probes and/or primers may be used in conjunction with any suitable nucleic acid analysis (e.g., a nucleic acid analysis method described herein). For example, probes and/or primers may be used in an amplification process (e.g., PCR, quantitative PCR), FISH (e.g., labeled FISH probes, labeled FISH probe pairs (e.g., with fluorophore and quencher)), microarray, nucleic acid capture, nucleic acid enrichment, nucleic acid sequencing, and the like. Oligonucleotides may be designed to hybridize to a portion or portions of a genome that is/are proximal to, adjacent to, overlapping, partially overlapping, or spanning a structural variant or portion thereof.

Oligonucleotides may include a probe or primer capable of hybridizing to a region of a first breakpoint and a region of a second breakpoint of a structural variant described herein. Accordingly, such probes and primers comprise a first sequence complementary to a receiving site in a structural variant and a second sequence complementary to a donor site in a structural variant. Such probes and primers are useful for detecting the presence, absence, or amount of a structural variant in a sample, for example, by way of hybridizing to the sample nucleic acid when the structural variant is present and not hybridizing to the sample nucleic acid when the structural variant is absent.

In some embodiments, an oligonucleotide comprises (i) a first polynucleotide identical to or complementary to a subsequence of 5 or more consecutive nucleotides in length within a region of a chromosome comprising a receiving site for a structural variant described herein, and (ii) a second polynucleotide identical to or complementary to a subsequence of about 5 or more consecutive nucleotides in length within a region of a chromosome comprising a donor site for a structural variant described herein. Such oligonucleotide can specifically hybridize (e.g., under stringent hybridization conditions) to a target sequence comprising the subsequence of (i) and the subsequence of (ii). Methods for hybridization reaction temperature condition optimization are known, and can be found, e.g., in Current Protocols in Molecular Biology, John Wiley & Sons, N.Y., 6.3.1-6.3.6 (1989). Aqueous and non-aqueous methods are described in the aforementioned reference and either can be used. Non-limiting examples of stringent hybridization conditions include, for example, hybridization in 6× sodium chloride/sodium citrate (SSC) at about 45° C., followed by one or more washes in 0.2×SSC, 0.1% SDS at 50° C. Another example of stringent hybridization conditions includes hybridization in 6× sodium chloride/sodium citrate (SSC) at about 45° C., followed by one or more washes in 0.2×SSC. 0.1% SDS at 55° C. A further example of stringent hybridization conditions includes hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions can include 0.5 M sodium phosphate, 7% SDS at 65° C., followed by one or more washes at 0.2×SSC, 1% SDS at 65° C. Stringent hybridization temperatures also can be altered (generally, lowered) with the addition of certain organic solvents, such as formamide for example. Organic solvents such as formamide can reduce the thermal stability of double-stranded polynucleotides, so that hybridization can be performed at lower temperatures, while still maintaining stringent conditions and extending the useful life of heat labile nucleic acids

In some embodiments, an oligonucleotide comprises (i) a first polynucleotide identical to or complementary to a subsequence of 5 or more consecutive nucleotides in length within a region of chromosome 9, where the region spans positions 5,471,000 to 5,480,000 or spans positions 5,475,001 to 5,476,000; and (ii) a second polynucleotide identical to or complementary to a subsequence of about 5 or more consecutive nucleotides in length within a region of chromosome 9, where the region spans positions 18,377,000 to 18,386,000 or spans positions 18,381,001 to 18,382,000. The oligonucleotide may specifically hybridize (e.g., under stringent hybridization conditions) to a target sequence comprising the subsequence of (i) of chromosome 9 and the subsequence of (ii) of chromosome 9.

Oligonucleotides may include a pair of probes or primers capable of hybridizing to a region of a first breakpoint and a region of a second breakpoint of a structural variant described herein. Accordingly, such probe and primer pairs comprise a first member complementary to a receiving site in a structural variant and a second member complementary to a donor site in a structural variant. Such probes and primers may be useful for detecting the presence or absence of a structural variant in a sample, for example, by way of hybridizing to the sample nucleic acid at specific locations when the structural variant is present and hybridizing to the sample nucleic acid at different locations when the structural variant is absent.

In some embodiments, a composition comprises (a) a first oligonucleotide comprising a first polynucleotide identical to or complementary to a subsequence of 5 or more consecutive nucleotides in length within a region of chromosome 9, where the region spans positions adjacent to the CD274 gene; and (b) a second oligonucleotide comprising a second polynucleotide identical to or complementary to a subsequence of about 5 or more consecutive nucleotides in length within a region of chromosome 9, where the region spans positions 18,377,000 to 18,386,000 or spans positions 18,381,001 to 18,382,000. In some embodiments, a composition comprises (a) a first oligonucleotide comprising a first polynucleotide identical to or complementary to a subsequence of 5 or more consecutive nucleotides in length within a region of chromosome 9, where the region spans positions adjacent to the CD273 gene; and (b) a second oligonucleotide comprising a second polynucleotide identical to or complementary to a subsequence of about 5 or more consecutive nucleotides in length within a region of chromosome 9, where the region spans positions 18,377,000 to 18,386,000 or spans positions 18,381,001 to 18,382,000. In some embodiments, a composition comprises (a) a first oligonucleotide comprising a first polynucleotide identical to or complementary to a subsequence of 5 or more consecutive nucleotides in length within a region of chromosome 9, where the region spans positions 5,471,000 to 5,480,000 or spans positions 5,475,001 to 5,476,000; and (b) a second oligonucleotide comprising a second polynucleotide identical to or complementary to a subsequence of about 5 or more consecutive nucleotides in length within a region of chromosome 9, where the region spans positions 18,377,000 to 18,386,000 or spans positions 18,381,001 to 18,382,000. The first oligonucleotide may specifically hybridize (e.g., under stringent hybridization conditions) to a target nucleic acid comprising the subsequence of chromosome 9 in (a). The second oligonucleotide may specifically hybridize (e.g., under stringent hybridization conditions) to a target nucleic acid comprising the subsequence of chromosome 9 in (b). In some embodiments, the first oligonucleotide specifically hybridizes (e.g., under stringent hybridization conditions) to a target nucleic acid comprising the subsequence of chromosome 9 in (a) and does not specifically hybridize to a target nucleic acid comprising the subsequence of chromosome 9 in (b). In some embodiments, the second oligonucleotide specifically hybridizes (e.g., under stringent hybridization conditions) to a target nucleic acid comprising the subsequence of chromosome 9 in (b) and does not specifically hybridize to a target nucleic acid comprising the subsequence of chromosome 9 in (a).

### Samples

Provided herein are methods and compositions for processing and/or analyzing nucleic acid. Nucleic acid utilized in methods and compositions described herein may be isolated from a sample obtained from a subject (e.g., a test subject). A subject can be any living or non-living organism, including but not limited to a human and a non-human animal. Any human or non-human animal can be selected, and may include, for example, mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a human. A subject may be a male or female. A subject may be any age (e.g., an embryo, a fetus, an infant, a child, an adult). A subject may be a cancer patient, a patient suspected of having cancer, a patient in remission, a patient with a family history of cancer, and/or a subject obtaining a cancer screen. In some embodiments, a subject is an adult patient. In some embodiments, a subject is a pediatric patient.

A nucleic acid sample may be isolated or obtained from any type of suitable biological specimen or sample (e.g., a test sample). A nucleic acid sample may be isolated or obtained from a single cell, a plurality of cells (e.g., cultured cells), cell culture media, conditioned media, a tissue, an organ, or an organism. In some embodiments, a nucleic acid sample is isolated or obtained from a cell(s), tissue, organ, and/or the like of an animal (e.g., an animal subject). In some instances, a nucleic acid sample may be obtained as part of a diagnostic analysis.

A sample or test sample may be any specimen that is isolated or obtained from a subject or part thereof (e.g., a human subject, a cancer patient, a tumor). Non-limiting examples of specimens include fluid or tissue from a subject, including, without limitation, blood or a blood product (e.g., serum, plasma, or the like), umbilical cord blood, chorionic villi, amniotic fluid, cerebrospinal fluid, spinal fluid, lavage fluid (e.g., bronchoalveolar, gastric, peritoneal, ductal, ear, arthroscopic), biopsy sample (e.g., from pre-implantation embryo; cancer biopsy), celocentesis sample, cells (blood cells, placental cells, embryo or fetal cells, fetal nucleated cells or fetal cellular remnants, normal cells, abnormal cells (e.g., cancer cells)) or parts thereof (e.g., mitochondrial, nucleus, extracts, or the like), washings of female reproductive tract, urine, feces, sputum, saliva, nasal mucous, prostate fluid, lavage, semen, lymphatic fluid, bile, tears, sweat, breast milk, breast fluid, the like or combinations thereof. In some embodiments, a biological sample is a cervical swab from a subject. A fluid or tissue sample from which nucleic acid is extracted may be acellular (e.g., cell-free). In some embodiments, a fluid or tissue sample may contain cellular elements or cellular remnants. In some embodiments, cancer cells may be included in the sample.

A sample can be a liquid sample. A liquid sample can comprise extracellular nucleic acid (e.g., circulating cell-free DNA). Examples of liquid samples include, but are not limited to, blood or a blood product (e.g., serum, plasma, or the like), urine, cerebrospinal fluid, saliva, sputum, biopsy sample (e.g., liquid biopsy for the detection of cancer), a liquid sample described above, the like or combinations thereof. In certain embodiments, a sample is a liquid biopsy, which generally refers to an assessment of a liquid sample from a subject for the presence, absence, progression or remission of a disease (e.g., cancer). A liquid biopsy can be used in conjunction with, or as an alternative to, a sold biopsy (e.g., tumor biopsy). In certain instances, extracellular nucleic acid is analyzed in a liquid biopsy.

In some embodiments, a biological sample may be blood, plasma or serum. The term “blood” encompasses whole blood, blood product or any fraction of blood, such as serum, plasma, buffy coat, or the like as conventionally defined. Blood or fractions thereof often comprise nucleosomes. Nucleosomes comprise nucleic acids and are sometimes cell-free or intracellular. Blood also comprises buffy coats. Buffy coats are sometimes isolated by utilizing a ficoll gradient. Buffy coats can comprise white blood cells (e.g., leukocytes, T-cells, B-cells, platelets, and the like). Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3 to 40 milliliters, between 5 to 50 milliliters) often is collected and can be stored according to standard procedures prior to or after preparation.

An analysis of nucleic acid found in a subject's blood may be performed using, e.g., whole blood, serum, or plasma. An analysis of tumor or cancer DNA found in a patient's blood, for example, may be performed using, e.g., whole blood, serum, or plasma. Methods for preparing serum or plasma from blood obtained from a subject (e.g., patient; cancer patient) are known. For example, a subject's blood (e.g., patient's blood; cancer patient's blood) can be placed in a tube containing EDTA or a specialized commercial product such as Cell-Free DNA BCT (Streck, Omaha, NE) or Vacutainer SST (Becton Dickinson, Franklin Lakes, N.J.) to prevent blood clotting, and plasma can then be obtained from whole blood through centrifugation. Serum may be obtained with or without centrifugation-following blood clotting. If centrifugation is used then it is typically, though not exclusively, conducted at an appropriate speed, e.g., 1,500-3,000 times g. Plasma or serum may be subjected to additional centrifugation steps before being transferred to a fresh tube for nucleic acid extraction. In addition to the acellular portion of the whole blood, nucleic acid may also be recovered from the cellular fraction, enriched in the buffy coat portion, which can be obtained following centrifugation of a whole blood sample from the subject and removal of the plasma.

A sample may be a tumor nucleic acid sample (i.e., a nucleic acid sample isolated from a tumor). The term “tumor” generally refers to neoplastic cell growth and proliferation, whether malignant or benign, and may include pre-cancerous and cancerous cells and tissues. The terms “cancer” and “cancerous” generally refer to the physiological condition in mammals that is typically characterized by unregulated cell growth/proliferation.

In some embodiments, a sample is a tissue sample, a cell sample, a blood sample, or a urine sample. In some embodiments, a sample comprises formalin-fixed, paraffin-embedded (FFPE) tissue. In some embodiments, a sample comprises frozen tissue. In some embodiments, a sample comprises peripheral blood. In some embodiments, a sample comprises blood obtained from bone marrow. In some embodiments, a sample comprises cells obtained from urine. In some embodiments, a sample comprises cell-free nucleic acid. In some embodiments, a sample comprises one or more tumor cells. In some embodiments, a sample comprises one or more circulating tumor cells. In some embodiments, a sample comprises a solid tumor. In some embodiments, a sample comprises a blood tumor.

### Cancers

In some embodiments, a subject has, or is suspected of having, a disease. In some embodiments, a subject has, or is suspected of having, cancer. In some embodiments, a subject has, or is suspected of having, a cancer associated with one or more genes and/or oncogenes described herein. For example, in some embodiments, a subject has, or is suspected of having, a cancer associated with one or more genes and/or oncogenes chosen from the CD274 gene, the CD273 gene, and the CD279 gene (the CD279 gene encodes the PD-1 receptor). In some embodiments, a subject has, or is suspected of having, a cancer associated with one or more structural variants described herein. In some embodiments, a subject has, or is suspected of having, a cancer associated with the CD274 gene. In some embodiments, a subject has, or is suspected of having, a cancer associated with one or more structural variants described herein. In some embodiments, a subject has, or is suspected of having, a cancer associated with the CD273 gene. In some embodiments, a subject has, or is suspected of having, a cancer associated with one or more structural variants described herein. In some embodiments, a subject has, or is suspected of having, a cancer associated with the CD274 gene and the CD273 gene.

Examples of cancer include, but are not limited to, carcinoma, lymphoma, blastoma, sarcoma, leukemia, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, a chordoma, a salivary gland carcinoma, kidney cancer, liver cancer, prostate cancer, vulval cancer, thyroid cancer, hepatic carcinoma, various types of head and neck cancer, and the like. In some embodiments, a cancer is a rare cancer. In some embodiments, a cancer is kidney cancer, breast cancer, colorectal cancer, gastric cancer, lung cancer, thyroid cancer, or testicular cancer. In some embodiments, a cancer is glioma. In some embodiments, a cancer is glioblastoma. In some embodiments, a cancer is a glioblastoma. In embodiments, a cancer is pediatric glioblastoma. In certain embodiments, the glioblastoma is a newly diagnosed. In embodiments, the glioblastoma is recurrent.

### Diagnosis and Treatment

In some embodiments, a method herein comprises providing a diagnosis and/or a likelihood of cancer in a subject. A diagnosis and/or likelihood of cancer may be provided when the presence of a structural variant described herein is detected. In some embodiments, a method herein comprises performing a further test (e.g., biopsy, blood test, imaging) to confirm a cancer diagnosis.

In some embodiments, a method herein comprises administering a treatment to a subject. A treatment may be administered to a subject when the presence of a structural variant described herein is detected. Suitable treatments may be determined by a physician and may include one or more modulators (e.g., activators, blockers) of one or more genes, proteins, oncogenes, oncoproteins (proteins encoded by oncogenes), and/or oncogene-related components associated with a detected structural variant.

An oncogene-related component generally refers to one or more components chosen from among (i) an oncogene, including exons, introns, and 5′ (upstream), e.g. promoter regions, or 3′ (downstream) regulatory elements; (ii) transcription products, mRNA, or cDNA; (iii) translation products, protein, gene products, or gene expression products, or homologs of, synthetic versions of, analogs of, receptors of, agonists to receptors of, antagonists to receptors of upstream pathway regulators of, or downstream pathway targets of translation products, protein, gene products, or gene expression products; and (iv) any component that could be considered by one skilled in the art as a target for a modulator (e.g., activator, blocker, drug, medicament).

The term “modulator” of a gene (or oncogene) or “gene/oncogene modulator” includes modulation of the gene/oncogene, modulation of a protein encoded by the gene/oncogene and/or modulation of a gene/oncogene—related component, such as a component of the pathway through which the gene/oncogene mediates its effects, a promoter, an enhancer, and the like.

A modulator generally refers to an agent that is capable of changing an activity (e.g., change in level and/or nature of an activity) of a component in a system, compared to a component's level and/or activity under otherwise comparable conditions when the modulator is absent. A modulator herein may refer to an agent that is capable of changing an activity (e.g., change in level and/or nature of an activity) of a gene, protein, an oncogene, oncoprotein, and/or oncogene-related component in a system compared to a gene's, a protein's, an oncogene's, oncoprotein's, and/or oncogene-related component's level and/or activity under otherwise comparable conditions when the modulator is absent. In some embodiments, a modulator is an activator, in that level and/or activity is increased in its presence as compared with that observed under otherwise comparable conditions when the modulator is absent. In some embodiments, a modulator is an inhibitor, in that level and/or activity is reduced in its presence as compared with otherwise comparable conditions when the modulator is absent. In some embodiments, a modulator interacts directly with a target component of interest. In some embodiments, a modulator interacts indirectly (e.g., directly with an intermediate agent that interacts with the target component) with a target component of interest. In some embodiments, a modulator affects the level of a target component of interest, as one non-limiting example, by impacting an upstream signaling pathway associated with the target component of interest. In some embodiments, a modulator affects an activity of a target component of interest without affecting a level of the target component, as one non-limiting example, by impacting a downstream signaling pathway associated with the target component of interest. In some embodiments, a modulator affects both level and activity of a target component of interest, such that an observed difference in activity is not entirely explained by or commensurate with an observed difference in level.

The term “modulator of [oncogene]” or “[oncogene] modulator” means “modulator of [oncogene], modulator of [onco]protein, and/or [oncogene]-related components” or “[oncogene], [onco]protein, and/or [oncogene]-related components modulator,” respectively, where [oncogene] can mean any oncogene identified herein.

In certain embodiments, the subject has a glioblastoma. In embodiments, the glioblastoma is a pediatric glioblastoma. In embodiments, the glioblastoma is a newly diagnosed glioblastoma. In certain embodiments, the glioblastoma is a recurrent glioblastoma. In embodiments, the glioblastoma, is glioblastoma multiforme.

In certain embodiments, the subject having cancer and identified as having a structural variant adjacent to the CD274 gene and/or adjacent to the CD273 gene is treated with a standard of care treatment for the cancer and, additionally, is treated with a treatment that inhibits a PD-1 receptor-mediated pathway and/or inhibits the interaction of the PD-1 receptor with PD-L1 and/or PD-L2. In embodiments, the subject having cancer and identified as having a structural variant adjacent to the CD274 gene is treated with a treatment that inhibits a PD-1 receptor-mediated pathway and/or inhibits the interaction of the PD-1 receptor with PD-L1 and/or PD-L2, instead of treatment with a standard of care treatment. The subject identified as having a structural variant adjacent to the CD274 gene, and treated with a treatment that inhibits a PD-1 receptor-mediated pathway and/or inhibits the interaction of the PD-1 receptor with PD-L1 and/or PD-L2, can be a subject that previously was undergoing a standard of care treatment or can be a subject not previously undergoing a standard of care treatment (i.e., the first line of treatment administered is a treatment that inhibits a PD-1 receptor-mediated pathway and/or inhibits the interaction of the PD-1 receptor with PD-L1 and/or PD-L2). Treatment regimens also can be modified, e.g., to incorporate portions of a standard of care treatment regimen along with a treatment that inhibits a PD-1 receptor-mediated pathway and/or inhibits the interaction of the PD-1 receptor with PD-L1 and/or PD-L2.

A “standard of care treatment,” or “standard treatment,” as used herein, is a treatment that generally is accepted by medical experts and/or health professionals as being the most appropriate for a certain type of disease in a certain setting and/or based on the age, gender, coexisting health conditions, stage of the disease, e.g., cancer, and the like. A standard of care treatment can, in certain aspects, be FDA approved or at a Phase II or greater stage of FDA approval (e.g., for experimental therapies in subjects selected for clinical trials). In certain embodiments, the subject treated with a standard of care treatment has a glioblastoma. In embodiments, the glioblastoma is a pediatric glioblastoma. In embodiments, the glioblastoma is a newly diagnosed glioblastoma. In certain embodiments, the glioblastoma is a recurrent glioblastoma. In embodiments, the glioblastoma, is glioblastoma multiforme.

Standard of care treatments for glioblastoma are known and are described, for example, in Glioblastoma (Edited by Steven De Vleeschouwer; Codon Publications, Brisbane, Australia (2017)) and in Liu et al., J. Cancer, 13(1): 354-363 (2022), the contents of each of which are incorporated in their entirety by reference herein, to the extent permitted by law. Standard of care treatments for a glioblastoma can include, but are not limited to, performing and/or administering one or more of the following: surgical tumor resection, radiotherapy, positron emission tomography (PET)-guided radiotherapy, positron emission tomography (PET)-guided dose escalated radiotherapy, laser interstitial thermal therapy, stereotactic radiosurgery (SRS), hypofractionated stereotactic radiotherapy (HFSRT), Tumor-Treating Fields (TTFields), chemoradiotherapy, brachytherapy, carmustine implantable wafers, temozolomide, vincristine, interferon, bevacizumab, onartuzumab, a nitrosourea, procarbazine, enzastaurin, teniposide, cytarabine, vincristine, irinotecan, carboplatin, dasatinib, temsirolimus, erlotinib, sorafenib, veliparib, galunisertib, cediranib, vorinostat, panobinostat, dianhydrogalactitol (VAL-083) and paclitaxel poliglumex (PPX).

In embodiments, the subject having cancer and identified as having a structural variant adjacent to the CD274 gene and/or adjacent to the CD273 gene is refractory to a standard of care treatment for the cancer, e.g., is refractory to a standard of care treatment for glioma. The term “refractory,” as used herein, means that progression of the disease is observed during or following one or more cycles, or a complete course, of a standard of care treatment.

In some embodiments, a method herein comprises predicting an outcome of a cancer treatment. An outcome of a cancer treatment may be predicted when the presence of a structural variant described herein is detected. For example, an outcome of a cancer treatment that includes a gene-specific modulator and/or an oncogene-specific modulator may be predicted when the presence of a structural variant associated with the gene and/or oncogene is detected.

In some embodiments, a sample from a subject is obtained over a plurality of time points. A plurality of time points may include time point over a number of days, weeks, months, and/or years. In some embodiments, a disease state is monitored over a plurality of time points. For example, a method to detect the presence, absence, or amount of a structural variant described herein may be performed over a plurality of time points to monitor the status of a disease (e.g., a disease (e.g., cancer) associated with the structural variant detected). In some embodiments, minimal residual disease (MRD) is monitored in a subject. Minimal residual disease (MRD) generally refers to cancer cells remaining after treatment that often cannot be detected by standard scans (e.g., X-ray, mammogram, computerized tomography (CT) scan, bone scan, magnetic resonance imaging (MRI), positron emission tomography (PET) scan, ultrasound) or tests (blood test, tissue biopsy, needle biopsy, liquid biopsy, endoscopic exam). Such cells can have the potential to cause a relapse of cancer in a subject. In some embodiments, a method herein can include detecting a presence of minimal residual disease (MRD) in a subject when a structural variant described herein is present. In some embodiments, a method herein comprises detecting an absence of minimal residual disease (MRD) in a subject when a structural variant described herein is absent. In some embodiments, a method herein comprises detecting an amount of a structural variant described herein in a sample. A level of minimal residual disease (MRD) in a subject may be determined according to an amount of structural variant detected in a sample.

Treatment that Inhibits a PD-1 Receptor-Mediated Pathway and/or Inhibits the Interaction of the PD-1 Receptor with PD-L1 and/or PD-L2

In the methods provided herein, subjects having a structural variant adjacent to the CD274 gene are selected for treatment and/or are treated with a treatment that inhibits a PD-1 receptor-mediated pathway and/or inhibits the interaction of the PD-1 receptor with PD-L1 and/or PD-L2. PD-1 is a monomeric type I immune inhibitory transmembrane receptor, mainly expressed in T cells, B cells, natural killer cells, and many other tumor-infiltrating lymphocytes. Binding of PD-1 to its ligand, PD-L1, can trigger an inhibitory signal, leading to reduced T-cell proliferation, and anti-tumor immunity. Engagement of PD-1 by another ligand, PD-L2, also dramatically inhibits T cell receptor (TCR)-mediated proliferation and cytokine production by CD4⁺ T cells. The significant immune suppression caused by the interaction of PD-1 with PD-L1 and PD-L2 leads to tumor immune escape and poor subject (patient) survival rates. A treatment that inhibits a PD-1 receptor-mediated pathway and/or inhibits the interaction of the PD-1 receptor with PD-L1 and/or PD-L2 can overcome the immune suppression and provide better outcomes for subjects having cancer.

The subjects selected for treatment and/or treated with a treatment that inhibits a PD-1 receptor-mediated pathway and/or inhibits the interaction of the PD-1 receptor with PD-L1 and/or PD-L2, according to the methods provided herein, can have a structural variant adjacent to the CD274 gene, which encodes the PD-L1 ligand, a structural variant adjacent to the CD273 gene, which encodes the PD-L2 ligand, or a structural variant adjacent to the CD274 gene and a structural variant adjacent to the CD273 gene. In embodiments, a structural variant is a translocation. The treatment can be administered with a standard of care treatment or a modification thereof or can be administered instead of a standard of care treatment. In embodiments, the subject having cancer and identified as having a structural variant adjacent to the CD274 gene, adjacent to the CD273 gene, or adjacent to the CD274 gene and adjacent to the CD273 gene, is refractory to a standard of care treatment for the cancer, e.g., is refractory to a standard of care treatment for glioma. In certain embodiments, the cancer is a glioblastoma. In embodiments, the glioblastoma is a pediatric glioblastoma. In embodiments, the glioblastoma is a newly diagnosed glioblastoma. In certain embodiments, the glioblastoma is a recurrent glioblastoma. In embodiments, the glioblastoma, is glioblastoma multiforme. In embodiments, the subject is refractory to a standard of care treatment for a glioblastoma.

A treatment that inhibits a PD-1 receptor-mediated pathway(s) and/or inhibits the interaction of the PD-1 receptor with PD-L1 and/or PD-L2 can include, but is not limited to, administering one or more agents selected from among atezolizumab, avelumab, balstilimab, cemiplimab, cemiplimab-rwlc, dostarlimab, dostarlimab-gxly, durvalumab, nivolumab, pembrolizumab, penpulimab, retifanlimab, sintilimab, pidilizumab, BMS-936559 (MDX-1105), AMP-224 fusion protein and MPDL33280A. Any agent or combination(s) thereof that inhibit the PD-1 receptor, that inhibits one or more components of PD-1 receptor-mediated pathway(s), that inhibits the PD-L1 and/or PD-L2 ligands, and/or inhibits the interaction of the PD-1 receptor with PD-L1 and/or PD-L2, can be administered in the methods provided herein. Non-limiting examples of an agent can include a small molecule, a nucleic acid (e.g., DNA, RNA such as mRNA or siRNA, PNA), and a protein, such as fusion protein, a modified PD-L1 and/or PD-L2 ligand, or an antibody (including bispecific antibodies, diabodies, and the like) or an antigen-binding fragment thereof.

Knowing the structures and mechanisms of action of PD-1 receptor-mediated pathway(s), and ligands/other components thereof, and given the knowledge regarding the structures and functions available agents that inhibit the pathway(s), the receptor, the ligands and/or other components, it is within the level of one of skill to design and produce additional agents that can inhibit a PD-1 receptor-mediated pathway(s) and/or inhibits the interaction of the PD-1 receptor with PD-L1 and/or PD-L2.

For example, when the agent is an antibody, such as a polyclonal antibody or a monoclonal antibody, it can be prepared using standard methods (see, e.g., Kohler et al., Nature 256:495-497 (1975); Kohler et al., Eur. J. Immunol. 6:511-519 (1976); and WO 02/46455). For example, to generate polyclonal antibodies, an immune response is elicited in a host animal, to an antigen of interest. Blood from the host animal is then collected and the serum fraction containing the secreted antibodies is separated from the cellular fraction, using methods known to those of skill in the art. To generate monoclonal antibodies, an animal is immunized by standard methods to produce antibody-secreting somatic cells. These cells then are removed from the immunized animal for fusion to myeloma cells. Somatic cells that can produce antibodies, particularly B cells, can be used for fusion with a myeloma cell line. These somatic cells can be derived from the lymph nodes, spleens and peripheral blood of primed animals. Specialized myeloma cell lines have been developed from lymphocytic tumors for use in hybridoma-producing fusion procedures (Kohler and Milstein, Eur. J. Immunol. 6:511-519 (1976); Shulman et al., Nature, 276:269-282 (1978); Volk et al., J. Virol., 42:220-227 (1982)). These cell lines have three useful properties. The first is they facilitate the selection of fused hybridomas from unfused and similarly indefinitely self-propagating myeloma cells by having enzyme deficiencies that render them incapable of growing in selective medium that support the growth of hybridomas. The second is they have the ability to produce antibodies and are incapable of producing endogenous light or heavy immunoglobulin chains. A third property is they efficiently fuse with other cells. Other methods for producing hybridomas and monoclonal antibodies are well known to those of skill in the art. It is routine to produce antibodies against any polypeptide, e.g., antigenic marker on an immune cell population.

Typically, monoclonal antibodies are developed in mice, rats or rabbits. The antibodies can be produced by immunizing an animal with an immunogenic amount of cells, cell extracts, or protein preparations that contain the desired epitope. The immunogen can be, but is not limited to, primary cells, cultured cell lines, cancerous cells, proteins, peptides, nucleic acids, or tissue. Cells used for immunization can be cultured for a period of time (e.g., at least 24 hours) prior to their use as an immunogen. Cells can be used as immunogens by themselves or in combination with a non-denaturing adjuvant, such as Ribi (see, e.g., Jennings, V. M. (1995) “Review of Selected Adjuvants Used in Antibody Production,” ILAR J. 37 (3): 119-125). In general, cells should be kept intact and preferably viable when used as immunogens. Intact cells can allow antigens to be better detected than ruptured cells by the immunized animal. Use of denaturing or harsh adjuvants, e.g., Freud's adjuvant, can rupture cells. The immunogen can be administered multiple times at periodic intervals such as, bi-weekly, or weekly, or can be administered in such a way as to maintain viability in the animal (e.g., in a tissue recombinant). Alternately, existing monoclonal antibodies and any other equivalent antibodies that are immunospecific for a desired pathogenic epitope can be sequenced and produced recombinantly by any means known in the art.

In aspects, an antibody can be sequenced, and the component polynucleotide sequences (or single sequence, in the case of ScFv) can then be cloned into a vector for expression or propagation. The polynucleotide sequence(s) encoding the antibody of interest can be maintained in a vector in a host cell and the host cell can then be expanded and frozen for future use. The polynucleotide sequence(s) of such antibodies can also be used for genetic manipulation to generate multispecific (e.g., bispecific, trispecific and tetraspecific) binding molecules as well as an affinity optimized, a chimeric antibody, a humanized antibody, and/or a caninized antibody, to improve the affinity, or other characteristics of the antibody. The general principle in humanizing an antibody involves retaining the basic sequence of the antigen-binding portion of the antibody such as 1, 2, 3, 4, 5 or all 6 of the CDR sequences, while swapping the non-human remainder of the antibody with human antibody sequences.

Other proteins that are therapeutic agents, such as modified PD-L1 or PD-L2 ligands or other components that are part of or modulate a PD-1 receptor-mediated pathway, can be produced by direct peptide synthesis, using, for example, well-known solid-phase techniques (see e.g., Stewart et al. (1969) Solid-Phase Peptide Synthesis, WH Freeman Co., San Francisco; Merrifield J (1963) J Am Chem Soc., 85:2149-2154). In vitro protein synthesis can be performed using manual techniques or by automation. Automated synthesis can be achieved, for example, using Applied Biosystems 431A Peptide Synthesizer (Perkin Elmer, Foster City CA) in accordance with the instructions provided by the manufacturer. Various fragments of a polypeptide can be chemically synthesized separately and combined using chemical methods. Such proteins also can be produced by recombinant means using well-known molecular biological methods of expressing proteins encoded by cloned DNA. In embodiments, the proteins can be mutated forms of a known therapeutic agent or can be mutated forms of a PD-L1 ligand, PD-L2 ligand, or other component of a PD-1 receptor-mediated pathway. Any method known in the art to effect mutation of any one or more amino acids in a target protein can be employed. Methods include standard site-directed or random mutagenesis of encoding nucleic acid molecules, or solid phase polypeptide synthesis methods. For example, nucleic acid molecules encoding a protein can be subjected to mutagenesis, such as random mutagenesis of the encoding nucleic acid, error-prone PCR, site-directed mutagenesis, overlap PCR, gene shuffling, or other recombinant methods. The nucleic acid encoding the polypeptides can then be introduced into a host cell to be expressed heterologously. In embodiments, the protein therapeutic agent can be a fusion protein or polypeptide. The fusion protein or polypeptide can be generated by direct chemical synthesis. The fusion protein or polypeptide also can be produced as a recombinant fusion polypeptide encoded by a nucleic acid sequence containing a coding sequence from one nucleic acid molecule and the coding sequence from another nucleic acid molecule in which the coding sequences are in the same reading frame such that when the fusion construct is transcribed and translated in a host cell, a fusion protein or polypeptide containing two or more different protein or polypeptide components is produced. The two different molecules can be adjacent in the construct or can be separated by a linker polypeptide that contains, e.g., 1, 2, 3, or more, but typically fewer than 10, 9, 8, 7, or 6 amino acids.

Typically, the agents (therapeutic agents) are administered in an amount that does not result in undesirable side effects of the subject (patient) being treated, or that minimizes or reduces the observed side effects and it is within the level of one of skill in the art to determine the appropriate dosages of these agents when administered singly, as a combination of two or more of the agent, or as a combination that includes all or part of a standard of care treatment regimen. Agents can be administered as a single dosage administration or as a multiple dosage administration. In certain aspects, an agent can be administered as a sustained release formulation. In aspects, the agents can be administered as an intravenous dose. The intravenous dose can be administered as a one-time treatment, or can be administered at intervals, such as, for example, once every 3 days, 4 days, 5 days, 6 days, 1 week, 2 weeks, 3 weeks, 1 month, 2 months, 3 months, or longer intervals. In aspects, the interval is 2 weeks. The dosage amount can range from about or 0.1 mg/kg to about or 100 mg/kg, such as, for example, about or 0.5 mg/kg to about or 50 mg/kg, about or 5 mg/kg to about or 50 mg/kg, about or 1 mg/kg to about or 20 mg/kg, about or 1 mg/kg to about or 100 mg/kg, about or 10 mg/kg to about or 80 mg/kg, or about or 50 mg/kg to about or 100 mg/kg or more; or at a dosage of about or 0.01 mg/m² to about or 800 mg/m² or more, such as for example, about or 0.01 mg/m², about or 0.1 mg/m², about or 0.5 mg/m², about or 1 mg/m², about or 5 mg/m², about or 10 mg/m², about or 15 mg/m², about or 20 mg/m², about or 25 mg/m², about or 30 mg/m², about or 35 mg/m², about or 40 mg/m², about or 45 mg/m², about or 50 mg/m², about or 100 mg/m², about or 150 mg/m², about or 200 mg/m², about or 250 mg/m², about or 300 mg/m², about or 400 mg/m², about or 500 mg/m², about or 600 mg/m² about or 700 mg/m².

Examples of dosing regimens and characteristics of agents that inhibit the interaction of the PD-1 receptor with PD-L1 and/or PD-L2 are provided in Table 1 below (reproduced from Chen et al., Frontiers in Immunol., Vol. 11: Article 1088 (May 2020), the contents of which are incorporated in their entirety by reference herein, to the extent permitted by law):

TABLE 1

| | Drug | | | | | | | |
|---|---|---|---|---|---|---|---|---|
| | Nivolumab | Pembrolizumab | Atezolizumab | | Avelumab | | Durvalumab | |
| | Subtypes | | | | | | | |
| | IgG4 | IgG4 | IgG1 | | IgG1 | | IgG1 | |
| Binding area | N-loop | C′D loop | CC′FG | | F and G | | the N-terminal | |
| | | | strands | | strands | | region, the CC′ | |
| | | | | | | | loop and the | |
| | | | | | | | CC′FG strands | |
| Buried surface | 1,487- | 1,774- | 2,106 | Å | 1,815 | Å | 1,624 | Å |
| | 1,932.5 Å | 2,126 Å | | | | | | |
| Affinity | 3.06 nM | 29 pM | 1.75 | nM | 0.046 | nM | 0.66 | nM |

| | | | | | |
|---|---|---|---|---|---|
| Degree of | Fully | Humanized | Fully | Fully | Fully |
| humanization | human | | human | human | human |
| Immunogenicity | 12.9% | 1.8% | NR | NR | 0.4% |
| Modification | S228P | S228P | Fc | — | Fc |
| | | | engineering | | engineering |
| Distribution | Spleen | Lung, liver, | NR | NR | NR |
| | | kidney, spleen | | | |
| Half life (days) | 26.7 | 25.8 | 27 | 6 | 17 |
| Dosage regimen | 3 mg/kg | 200 mg | 1,200 mg | 10 mg/kg | 10 mg/kg |
| | q2 w* | q3 w | q3 w | q2 w | q2 w |

*q2 w = every 2 weeks; q3 w = every 3 weeks

### Certain Implementations

Following are non-limiting examples of certain implementations of the technology.
A1. A method of treating a subject that has, or is suspected of having, cancer, the method comprising:
   a) identifying and/or selecting a subject comprising a structural variant in the genome of the subject, wherein the location of the structural variant, or a breakpoint of the structural variant, is adjacent to the CD274 gene; and
   b) if the subject has cancer, treating the subject so identified and/or selected with a treatment that inhibits a PD-1 receptor-mediated pathway and/or inhibits the interaction of the PD-1 receptor with PD-L1 and/or PD-L2.
A2. The method of embodiment A1, wherein a breakpoint of the structural variant maps to a location between positions 5,471,000 to 5,480,000 on chromosome 9, wherein the positions are in reference to an HG38 human reference genome.
A3. The method of embodiment A1 or A2, wherein a breakpoint of the structural variant maps to a location between positions 5,475,001 to 5,476,000 on chromosome 9, wherein the positions are in reference to an HG38 human reference genome.
A4. The method of any one of embodiments A1 to A3, wherein the adjacent location of a breakpoint of the structural variant is at a distance of about 4,000 base pairs from the CD274 gene, wherein the distance is measured from the 3′ end of the CD274 gene.
A5. The method of any one of embodiments A1 to A4, wherein the adjacent location of a breakpoint of the structural variant is at a distance of about 4,400 base pairs from the CD274 gene, wherein the distance is measured from the 3′ end of the CD274 gene.
A6. The method of any one of embodiments A1 to A5, wherein the adjacent location of a breakpoint of the structural variant is at a distance of between about 4,000 base pairs to about 30,000 base pairs from the CD274 gene, wherein the closest distance of about 4,000 base pairs is measured from the 3′ end of the CD274 gene, and the farthest distance of about 30,000 base pairs from the CD274 gene, is measured from the 5′ end of the CD274 gene.
A7. The method of any one of embodiments A1 to A6, wherein the adjacent location of the structural variant is at a distance of between about 4,400 base pairs to about 25,000 base pairs from the CD274 gene, wherein the closest distance of about 4,400 base pairs is measured from the 3′ end of the CD274 gene, and the farthest distance of about 25,000 base pairs from the CD274 gene, is measured from the 5′ end of the CD274 gene.
A8. The method of any one of embodiments A1 to A7, wherein the adjacent location of the structural variant is at a distance of about 4,447 base pairs from the CD274 gene, wherein the distance is measured from the 3′ end of the CD274 gene.
A9. The method of any one of embodiments A1 to A8, wherein the adjacent location of the structural variant is at a distance of between about 4,447 base pairs to about 24,459 base pairs from the CD274 gene, wherein the closest distance of about 4,447 base pairs is measured from the 3′ end of the CD274 gene, and the farthest distance of about 24,459 base pairs from the CD274 gene, is measured from the 5′ end of the CD274 gene.
A9.1. The method of embodiment A9, wherein the distance is a linear distance.
A9.2. A method of treating a subject that has, or is suspected of having, cancer, the method comprising:
   a) identifying and/or selecting a subject comprising a structural variant in the genome of the subject, wherein the location of the structural variant, or a breakpoint of the structural variant, is adjacent to the CD273 gene; and
   b) if the subject has cancer, treating the subject so identified and/or selected with a treatment that inhibits a PD-1 receptor-mediated pathway and/or inhibits the interaction of the PD-1 receptor with PD-L1 and/or PD-L2.
A9.3. The method of any one of embodiments A1 to A9.1, further comprising:
   a) identifying and/or selecting a subject comprising a structural variant in the genome of the subject, wherein the location of the structural variant, or a breakpoint of the structural variant, is adjacent to the CD273 gene; and
   b) if the subject has cancer, treating the subject so identified and/or selected with a treatment that inhibits a PD-1 receptor-mediated pathway and/or inhibits the interaction of the PD-1 receptor with PD-L1 and/or PD-L2.
A9.4. The method of embodiment A9.2 or A9.3, wherein the adjacent location of the structural variant, and/or a breakpoint of the structural variant, is at a distance of about 30,000 base pairs to about 35,000 base pairs from the CD273 gene, wherein the distance is measured from the 5′ end of the CD273 gene.
A9.5 The method of embodiment A9.4, wherein the adjacent location of the structural variant and/or a breakpoint of the structural variant, is at a closest distance of between about 33,000 base pairs to about 35,000 base pairs from the CD273 gene.
A9.6. The method of embodiment A9.5, wherein the adjacent location of the structural variant and/or a breakpoint of the structural variant is at a closest distance of about 34,531 base pairs from the CD273 gene.
A9.7. The method of any one of embodiments A9.4 to A9.6, wherein the distance is a linear distance.
A10. The method of any one of embodiments A1 to A9.7, wherein the structural variant comprises an ectopic portion of genomic DNA from positions 18,377,000 to 18,386,000 on chromosome 9.
A11. The method of any one of embodiments A1 to A10, wherein the structural variant comprises an ectopic portion of genomic DNA from positions 18,381,001 to 18,382,000 on chromosome 9.
A12. The method of any one of embodiments A1 to A11, wherein the adjacent location is on the p arm of chromosome 9.
A13. The method of any one of embodiments A10 to A12, wherein the ectopic portion of genomic DNA is on the p arm of chromosome 9.
A14. The method of any one of embodiments A1 to A13, wherein the cancer is a rare cancer.
A15. The method of any one of embodiment A1 to A14, wherein the cancer is kidney cancer, a cancer of the central nervous system (CNS), breast cancer, colorectal cancer, gastric cancer, lung cancer, thyroid cancer, or testicular cancer.
A16. The method of embodiment A15, wherein the cancer is a cancer of the central nervous system (CNS).
A17. The method of any one of embodiments A14 to A16, wherein the cancer is a brain cancer.
A18. The method of embodiment A17, wherein the cancer is a glioma.
A19. The method of embodiment A18, wherein the cancer is a glioblastoma.
A20. The method of embodiment A19, wherein the cancer is glioblastoma multiforme.
A21. The method of embodiment A19 or A20, wherein the cancer is pediatric glioblastoma.
A22. The method of any one of embodiments A19 to A21, wherein the glioblastoma is a newly diagnosed glioblastoma.
A23. The method of any one of embodiments A19 to A21, wherein the glioblastoma is a recurrent glioblastoma.
A24. The method of any one of embodiments A19 to A23, wherein the subject is refractory to a standard of care treatment for glioblastoma.
A25. The method of embodiment A24, wherein the standard of care treatment comprises performing and/or administering one or more of the following: surgical tumor resection, radiotherapy, positron emission tomography (PET)-guided radiotherapy, positron emission tomography (PET)-guided dose escalated radiotherapy, laser interstitial thermal therapy, stereotactic radiosurgery (SRS), hypofractionated stereotactic radiotherapy (HFSRT), Tumor-Treating Fields (TTFields), chemoradiotherapy, brachytherapy, carmustine implantable wafers, temozolomide, vincristine, interferon, bevacizumab, onartuzumab, a nitrosourea, procarbazine, enzastaurin, teniposide, cytarabine, vincristine, irinotecan, carboplatin, dasatinib, temsirolimus, erlotinib, sorafenib, veliparib, galunisertib, cediranib, vorinostat, panobinostat, dianhydrogalactitol (VAL-083) and paclitaxel poliglumex (PPX).
A26. The method of embodiment A24 or A25, wherein the standard of care treatment comprises performing and/or administering surgical tumor resection, radiotherapy and temozolomide.
A27. The method of any one of embodiments A24 to A26, wherein the standard of care treatment comprises carmustine implantable wafers.
A28. The method of any one of embodiments A24 to A27, wherein the standard of care treatment comprises administering bevacizumab.
A29. The method of any one of embodiments A24 to A28, wherein the standard of care treatment comprises administering a nitrosourea selected from among carmustine, lomustine, nimustine, nimustine hydrochloride and fotemustine.
A30. The method of any one of embodiments A24 to A29, wherein the standard of care treatment comprises administering temozolomide and bevacizumab.
A31. The method of any one of embodiments A24 to A30, wherein the standard of care treatment comprises performing and/or administering stereotactic radiosurgery (SRS) and/or hypofractionated stereotactic radiotherapy (HFSRT) and temozolomide.
A32. The method of any one of embodiment A1 to A31, wherein the structural variant comprises one or more of a translocation, inversion, insertion, deletion, and duplication.
A33. The method of any one of embodiments A1 to A32, wherein the structural variant comprises a microduplication and/or a microdeletion.
A34. The method of any one of embodiments A1 to A33, wherein identifying and/or selecting a subject comprising a structural variant in the genome of the subject comprises:
   1) performing a nucleic acid analysis on a sample obtained from a subject; and
   2) detecting whether the structural variant is present or absent in the sample according to the analysis in 1).
A35. The method of embodiment A34, wherein the nucleic acid analysis in 1) comprises a method that preserves spatial-proximal contiguity information.
A36. The method of embodiment A34 or A35, wherein the nucleic acid analysis in 1) comprises one or more of polymerase chain reaction (PCR), targeted sequencing, microarray, and fluorescence in situ hybridization (FISH).
A37. The method of any one of embodiments A34 to A36, wherein the nucleic acid analysis in 1) comprises generating proximity ligated nucleic acid molecules.
A38. The method of embodiment A37, wherein the nucleic acid analysis in 1) further comprises sequencing the proximity ligated nucleic acid molecules.
A39. The method of any one of embodiments A1 to A38, wherein the subject is a human.
A40. The method of embodiment A39, wherein the subject is an adult patient.
A41. The method of embodiment A39, wherein the subject is a pediatric patient.
A42. The method of any one of embodiments A34 to A41, wherein the sample is a tissue sample, a cell sample, a blood sample, or a urine sample.
A43. The method of any one of embodiments A34 to A42, wherein the sample comprises FFPE tissue.
A44. The method of any one of embodiments A34 to A43, wherein the sample comprises frozen tissue.
A45. The method of any one of embodiments A34 to A44, wherein the sample comprises peripheral blood.
A46. The method of any one of embodiments A34 to A45, wherein the sample comprises blood obtained from bone marrow.
A47. The method of any one of embodiments A34 to A44, wherein the sample comprises cells obtained from urine.
A48. The method of any one of embodiments A34 to A47, wherein the sample comprises cell-free nucleic acid.
A49. The method of any one of embodiments A34 to A48, wherein the sample comprises one or more tumor cells.
A50. The method of any one of embodiments A34 to A49, wherein the sample comprises one or more circulating tumor cells.
A51. The method of any one of embodiments A34 to A50, wherein the sample comprises or is obtained from a solid tumor.
A52. The method of any one of embodiments A34 to A51, wherein the sample comprises or is obtained from a brain tumor.
A53. The method of embodiment A52, wherein the brain tumor is a glioblastoma.
A54. The method of any one of embodiments A1 to A53, wherein the treatment that inhibits a PD-1 receptor-mediated pathway and/or inhibits the interaction of the PD-1 receptor with PD-L1 and/or PD-L2 comprises immunotherapy.
A55. The method of any one of embodiments A1 to A54, wherein the treatment that inhibits a PD-1 receptor-mediated pathway and/or inhibits the interaction of the PD-1 receptor with PD-L1 and/or PD-L2 is selected from a treatment comprising administering one or more of atezolizumab, avelumab, balstilimab, cemiplimab, cemiplimab-rwlc, dostarlimab, dostarlimab-gxly, durvalumab, nivolumab, pembrolizumab, penpulimab, retifanlimab, sintilimab, pidilizumab, BMS-936559 (MDX-1105), AMP-224 fusion protein and MPDL33280A.
A56. The method of embodiment A55, wherein the treatment that inhibits a PD-1 receptor-mediated pathway and/or inhibits the interaction of the PD-1 receptor with PD-L1 and/or PD-L2 comprises administering pembrolizumab.
A57. The method of any one of embodiments A1 to A56, wherein the treatment that inhibits a PD-1 receptor-mediated pathway and/or inhibits the interaction of the PD-1 receptor with PD-L1 and/or PD-L2 is administered in addition to a standard of care treatment for the cancer.
A58. The method of any one of embodiments A1 to A56, wherein the treatment that inhibits a PD-1 receptor-mediated pathway and/or inhibits the interaction of the PD-1 receptor with PD-L1 and/or PD-L2 is administered instead of a standard of care treatment for the cancer.
A59. The method of embodiment A57 or A58, wherein the subject is refractory to the standard of care treatment.
A60. The method of any one of embodiments A1 to A59, further comprising predicting an outcome of a treatment that inhibits a PD-1 receptor-mediated pathway and/or inhibits the interaction of the PD-1 receptor with PD-L1 and/or PD-L2 when the structural variant is present.
B1. A method of selecting a subject having cancer for treatment with an agent that inhibits a PD-1 receptor-mediated pathway and/or inhibits the interaction of the PD-1 receptor with PD-L1 and/or PD-L2, the method comprising:
   a) determining whether the subject comprises a structural variant in the genome of the subject, wherein the location of the structural variant, or a breakpoint of the structural variant, is adjacent to the CD274 gene; and
   b) if the structural variant, or a breakpoint of the structural variant, is identified in a), selecting the subject for treatment with an agent that inhibits a PD-1 receptor-mediated pathway and/or inhibits the interaction of the PD-1 receptor with PD-L1 and/or PD-L2.
B2. The method of embodiment B1, wherein a breakpoint of the structural variant maps to a location between positions 5,471,000 to 5,480,000 on chromosome 9, wherein the positions are in reference to an HG38 human reference genome.
B3. The method of embodiment B1 or B2, wherein a breakpoint of the structural variant maps to a location between positions 5,475,001 to 5,476,000 on chromosome 9, wherein the positions are in reference to an HG38 human reference genome.
B4. The method of any one of embodiments B1 to B3, wherein the adjacent location of the structural variant is at a distance of about 4,000 base pairs from the CD274 gene, wherein the distance is measured from the 3′ end of the CD274 gene.
B5. The method of any one of embodiments B1 to B4, wherein the adjacent location of the structural variant is at a distance of about 4,400 base pairs from the CD274 gene, wherein the distance is measured from the 3′ end of the CD274 gene.
B6. The method of any one of embodiments B1 to B5, wherein the adjacent location of the structural variant is at a distance of between about 4,000 base pairs to about 30,000 base pairs from the CD274 gene, wherein the distance is measured from the 3′ end of the CD274 gene.
B6. The method of any one of embodiments B1 to B5, wherein the adjacent location of a breakpoint of the structural variant is at a distance of between about 4,000 base pairs to about 30,000 base pairs from the CD274 gene, wherein the closest distance of about 4,000 base pairs is measured from the 3′ end of the CD274 gene, and the farthest distance of about 30,000 base pairs from the CD274 gene, is measured from the 5′ end of the CD274 gene.
B7. The method of any one of embodiments B1 to B6, wherein the adjacent location of the structural variant is at a distance of between about 4,400 base pairs to about 25,000 base pairs from the CD274 gene, wherein the closest distance of about 4,400 base pairs is measured from the 3′ end of the CD274 gene, and the farthest distance of about 25,000 base pairs from the CD274 gene, is measured from the 5′ end of the CD274 gene.
B8. The method of any one of embodiments B1 to B7, wherein the adjacent location of the structural variant is at a distance of about 4,447 base pairs from the CD274 gene, wherein the distance is measured from the 3′ end of the CD274 gene.
B9. The method of any one of embodiments B1 to B8, wherein the adjacent location of the structural variant is at a distance of between about 4,447 base pairs to about 24,459 base pairs from the CD274 gene, wherein the closest distance of about 4,447 base pairs is measured from the 3′ end of the CD274 gene, and the farthest distance of about 24,459 base pairs from the CD274 gene, is measured from the 5′ end of the CD274 gene.
B9.1. The method of embodiment B9, wherein the distance is a linear distance.
B9.2. A method of selecting a subject having cancer for treatment with an agent that inhibits a PD-1 receptor-mediated pathway and/or inhibits the interaction of the PD-1 receptor with PD-L1 and/or PD-L2, the method comprising:
   a) determining whether the subject comprises a structural variant in the genome of the subject, wherein the location of the structural variant, or a breakpoint of the structural variant, is adjacent to the CD273 gene; and
   b) if the structural variant, or a breakpoint of the structural variant, is identified in a), selecting the subject for treatment with an agent that inhibits a PD-1 receptor-mediated pathway and/or inhibits the interaction of the PD-1 receptor with PD-L1 and/or PD-L2.
B9.3. The method of any one of embodiments B1 to B9.1, further comprising:
   a) determining whether the subject comprises a structural variant in the genome of the subject, wherein the location of the structural variant, or a breakpoint of the structural variant, is adjacent to the CD273 gene; and
   b) if the structural variant, or a breakpoint of the structural variant, is identified in a), selecting the subject for treatment with an agent that inhibits a PD-1 receptor-mediated pathway and/or inhibits the interaction of the PD-1 receptor with PD-L1 and/or PD-L2.
B9.4 The method of embodiment B9.2 or B9.3, wherein the adjacent location of the structural variant, and/or a breakpoint of the structural variant, is at a distance of about 30,000 base pairs to about 35,000 base pairs from the CD273 gene, wherein the distance is measured from the 5′ end of the CD273 gene.
B9.5 The method of embodiment B9.4, wherein the adjacent location of the structural variant and/or a breakpoint of the structural variant, is at a closest distance of between about 33,000 base pairs to about 35,000 base pairs from the CD273 gene.
B9.6 The method of embodiment B9.5, wherein the adjacent location of the structural variant and/or a breakpoint of the structural variant is at a closest distance of about 34,531 base pairs from the CD273 gene.
B9.7 The method of any one of embodiments B9.4 to B9.6, wherein the distance is a linear distance.
B10. The method of any one of embodiments B1 to B9.7, wherein the structural variant comprises an ectopic portion of genomic DNA from positions 18,377,000 to 18,386,000 on chromosome 9.
B11. The method of any one of embodiments B1 to B10, wherein the structural variant comprises an ectopic portion of genomic DNA from positions 18,381,001 to 18,382,000 on chromosome 9.
B12. The method of any one of embodiments B1 to B11, wherein the adjacent location is on the p arm of chromosome 9.
B13. The method of any one of embodiments B10 to B12, wherein the ectopic portion of genomic DNA is on the p arm of chromosome 9.
B14. The method of any one of embodiments B1 to B13, wherein the cancer is a rare cancer.
B15. The method of any one of embodiment B1 to B14, wherein the cancer is kidney cancer, a cancer of the central nervous system (CNS), breast cancer, colorectal cancer, gastric cancer, lung cancer, thyroid cancer, or testicular cancer.
B16. The method of embodiment B15, wherein the cancer is a cancer of the central nervous system (CNS).
B17. The method of any one of embodiments B14 to B16, wherein the cancer is a brain cancer.
B18. The method of embodiment B17, wherein the cancer is a glioma.
B19. The method of embodiment B18, wherein the cancer is a glioblastoma.
B20. The method of embodiment B19, wherein the cancer is glioblastoma multiforme.
B21. The method of embodiment B19 or B20, wherein the cancer is pediatric glioblastoma.
B22. The method of any one of embodiments B19 to B21, wherein the glioblastoma is a newly diagnosed glioblastoma.
B23. The method of any one of embodiments B19 to B21, wherein the glioblastoma is a recurrent glioblastoma.
B24. The method of any one of embodiments B19 to B23, wherein the subject is refractory to a standard of care treatment for glioblastoma.
B25. The method of embodiment B24, wherein the standard of care treatment comprises performing and/or administering one or more of the following: surgical tumor resection, radiotherapy, positron emission tomography (PET)-guided radiotherapy, positron emission tomography (PET)-guided dose escalated radiotherapy, laser interstitial thermal therapy, stereotactic radiosurgery (SRS), hypofractionated stereotactic radiotherapy (HFSRT), Tumor-Treating Fields (TTFields), chemoradiotherapy, brachytherapy, carmustine implantable wafers, temozolomide, vincristine, interferon, bevacizumab, onartuzumab, a nitrosourea, procarbazine, enzastaurin, teniposide, cytarabine, vincristine, irinotecan, carboplatin, dasatinib, temsirolimus, erlotinib, sorafenib, veliparib, galunisertib, cediranib, vorinostat, panobinostat, dianhydrogalactitol (VAL-083) and paclitaxel poliglumex (PPX).
B26. The method of embodiment B24 or B25, wherein the standard of care treatment comprises performing and/or administering surgical tumor resection, radiotherapy and temozolomide.
B27. The method of any one of embodiments B24 to B26, wherein the standard of care treatment comprises carmustine implantable wafers.
B28. The method of any one of embodiments B24 to B27, wherein the standard of care treatment comprises administering bevacizumab.
B29. The method of any one of embodiments B24 to B28, wherein the standard of care treatment comprises administering a nitrosourea selected from among carmustine, lomustine, nimustine, nimustine hydrochloride and fotemustine.
B30. The method of any one of embodiments B24 to B29, wherein the standard of care treatment comprises administering temozolomide and bevacizumab.
B31. The method of any one of embodiments B24 to B30, wherein the standard of care treatment comprises performing and/or administering stereotactic radiosurgery (SRS) and/or hypofractionated stereotactic radiotherapy (HFSRT) and temozolomide.
B32. The method of any one of embodiment B1 to B31, wherein the structural variant comprises one or more of a translocation, inversion, insertion, deletion, and duplication.
B33. The method of any one of embodiments B1 to B32, wherein the structural variant comprises a microduplication and/or a microdeletion.
B34. The method of any one of embodiments B1 to B33, wherein determining whether the subject comprises a structural variant in the genome of the subject comprises:
1) performing a nucleic acid analysis on a sample obtained from a subject; and
2) detecting whether the structural variant is present or absent in the sample according to the analysis in 1).
B35. The method of embodiment B34, wherein the nucleic acid analysis in 1) comprises a method that preserves spatial-proximal contiguity information.
B36. The method of embodiment B34 or B35, wherein the nucleic acid analysis in 1) comprises one or more of polymerase chain reaction (PCR), targeted sequencing, microarray, and fluorescence in situ hybridization (FISH).
B37. The method of any one of embodiments B34 to B36, wherein the nucleic acid analysis in 1) comprises generating proximity ligated nucleic acid molecules.
B38. The method of embodiment B37, wherein the nucleic acid analysis in 1) further comprises sequencing the proximity ligated nucleic acid molecules.
B39. The method of any one of embodiments B1 to B38, wherein the subject is a human.
B40. The method of embodiment B39, wherein the subject is an adult patient.
B41. The method of embodiment B39, wherein the subject is a pediatric patient.
B42. The method of any one of embodiments B34 to B41, wherein the sample is a tissue sample, a cell sample, a blood sample, or a urine sample.
B43. The method of any one of embodiments B34 to B42, wherein the sample comprises FFPE tissue.
B44. The method of any one of embodiments B34 to B43, wherein the sample comprises frozen tissue.
B45. The method of any one of embodiments B34 to B44, wherein the sample comprises peripheral blood.
B46. The method of any one of embodiments B34 to B45, wherein the sample comprises blood obtained from bone marrow.
B47. The method of any one of embodiments B34 to B44, wherein the sample comprises cells obtained from urine.
B48. The method of any one of embodiments B34 to B47, wherein the sample comprises cell-free nucleic acid.
B49. The method of any one of embodiments B34 to B48, wherein the sample comprises one or more tumor cells.
B50. The method of any one of embodiments B34 to B49, wherein the sample comprises one or more circulating tumor cells.
B51. The method of any one of embodiments B34 to B50, wherein the sample comprises or is obtained from a solid tumor.
B52. The method of any one of embodiments B34 to B51, wherein the sample comprises or is obtained from a brain tumor.
B53. The method of embodiment B52, wherein the brain tumor is a glioblastoma.
B54. The method of any one of embodiments B1 to B53, further comprising, administering the treatment that inhibits a PD-1 receptor-mediated pathway and/or inhibits the interaction of the PD-1 receptor with PD-L1 and/or PD-L2, to the selected subject.
B55. The method of embodiment B54, wherein the treatment comprises immunotherapy.
B56. The method of any one of embodiments B1 to B55, wherein the treatment that inhibits a PD-1 receptor-mediated pathway and/or inhibits the interaction of the PD-1 receptor with PD-L1 and/or PD-L2 is selected from a treatment comprising administering one or more of atezolizumab, avelumab, balstilimab, cemiplimab, cemiplimab-rwlc, dostarlimab, dostarlimab-gxly, durvalumab, nivolumab, pembrolizumab, penpulimab, retifanlimab, sintilimab, pidilizumab, BMS-936559 (MDX-1105), AMP-224 fusion protein and MPDL33280A.
B57. The method of embodiment B56, wherein the treatment that inhibits a PD-1 receptor-mediated pathway and/or inhibits the interaction of the PD-1 receptor with PD-L1 and/or PD-L2 comprises administering pembrolizumab.
B58. The method of any one of embodiments B1 to B57, wherein the treatment that inhibits a PD-1 receptor-mediated pathway and/or inhibits the interaction of the PD-1 receptor with PD-L1 and/or PD-L2 is administered in addition to a standard of care treatment for the cancer.
B59. The method of any one of embodiments B1 to B57, wherein the treatment that inhibits a PD-1 receptor-mediated pathway and/or inhibits the interaction of the PD-1 receptor with PD-L1 and/or PD-L2 is administered instead of a standard of care treatment for the cancer.
B60. The method of embodiment B58 or B59, wherein the subject is refractory to the standard of care treatment.
B61. The method of any one of embodiments B1 to B60, further comprising predicting an outcome of a treatment that inhibits a PD-1 receptor-mediated pathway and/or inhibits the interaction of the PD-1 receptor with PD-L1 and/or PD-L2 when the structural variant is present.
C1. A method of screening a subject having cancer for potential responsiveness to treatment with an agent that inhibits a PD-1 receptor-mediated pathway and/or inhibits the interaction of the PD-1 receptor with PD-L1 and/or PD-L2, the method comprising:
   a) determining whether the subject comprises a structural variant in the genome of the subject, wherein the location of the structural variant, or a breakpoint of the structural variant, is adjacent to the CD274 gene; and
   b) if the structural variant, or a breakpoint of the structural variant, is identified in a), identifying the subject as potentially responsive to treatment with an agent that inhibits a PD-1 receptor-mediated pathway and/or inhibits the interaction of the PD-1 receptor with PD-L1 and/or PD-L2.
C2. The method of embodiment C1, wherein a breakpoint of the structural variant maps to a location between positions 5,471,000 to 5,480,000 on chromosome 9, wherein the positions are in reference to an HG38 human reference genome.
C3. The method of embodiment C1 or C2, wherein a breakpoint of the structural variant maps to a location between positions 5,475,001 to 5,476,000 on chromosome 9, wherein the positions are in reference to an HG38 human reference genome.
C4. The method of any one of embodiments C1 to C3, wherein the adjacent location of the structural variant is at a distance of about 4,000 base pairs from the CD274 gene, wherein the distance is measured from the 3′ end of the CD274 gene.
C5. The method of any one of embodiments C1 to C4, wherein the adjacent location of the structural variant is at a distance of about 4,400 base pairs from the CD274 gene, wherein the distance is measured from the 3′ end of the CD274 gene.
C6. The method of any one of embodiments C1 to C5, wherein the adjacent location of a breakpoint of the structural variant is at a distance of between about 4,000 base pairs to about 30,000 base pairs from the CD274 gene, wherein the closest distance of about 4,000 base pairs is measured from the 3′ end of the CD274 gene, and the farthest distance of about 30,000 base pairs from the CD274 gene, is measured from the 5′ end of the CD274 gene.
C7. The method of any one of embodiments C1 to C6, wherein the adjacent location of the structural variant is at a distance of between about 4,400 base pairs to about 25,000 base pairs from the CD274 gene, wherein the closest distance of about 4,400 base pairs is measured from the 3′ end of the CD274 gene, and the farthest distance of about 25,000 base pairs from the CD274 gene, is measured from the 5′ end of the CD274 gene.
C8. The method of any one of embodiments C1 to C7, wherein the adjacent location of the structural variant is at a distance of about 4,447 base pairs from the CD274 gene, wherein the distance is measured from the 3′ end of the CD274 gene.
C9. The method of any one of embodiments C1 to C8, wherein the adjacent location of the structural variant is at a distance of between about 4,447 base pairs to about 24,459 base pairs from the CD274 gene, wherein the closest distance of about 4,447 base pairs is measured from the 3′ end of the CD274 gene, and the farthest distance of about 24,459 base pairs from the CD274 gene, is measured from the 5′ end of the CD274 gene.
C9.1. The method of embodiment C9, wherein the distance is a linear distance.
C9.2. A method of screening a subject having cancer for potential responsiveness to treatment with an agent that inhibits a PD-1 receptor-mediated pathway and/or inhibits the interaction of the PD-1 receptor with PD-L1 and/or PD-L2, the method comprising:
   a) determining whether the subject comprises a structural variant in the genome of the subject, wherein the location of the structural variant, or a breakpoint of the structural variant, is adjacent to the CD273 gene; and
   b) if the structural variant, or a breakpoint of the structural variant, is identified in a), identifying the subject as potentially responsive to treatment with an agent that inhibits a PD-1 receptor-mediated pathway and/or inhibits the interaction of the PD-1 receptor with PD-L1 and/or PD-L2.
C9.3. The method of any one of embodiments C1 to C9.1, further comprising:
   a) determining whether the subject comprises a structural variant in the genome of the subject, wherein the location of the structural variant, or a breakpoint of the structural variant, is adjacent to the CD273 gene; and
   b) if the structural variant, or a breakpoint of the structural variant, is identified in a), identifying the subject as potentially responsive to treatment with an agent that inhibits a PD-1 receptor-mediated pathway and/or inhibits the interaction of the PD-1 receptor with PD-L1 and/or PD-L2.
C9.4 The method of embodiment C9.2 or C9.3, wherein the adjacent location of the structural variant, and/or a breakpoint of the structural variant, is at a distance of about 30,000 base pairs to about 35,000 base pairs from the CD273 gene, wherein the distance is measured from the 5′ end of the CD273 gene.
C9.5 The method of embodiment C9.4, wherein the adjacent location of the structural variant and/or a breakpoint of the structural variant, is at a closest distance of between about 33,000 base pairs to about 35,000 base pairs from the CD273 gene.
C9.6 The method of embodiment C9.5, wherein the adjacent location of the structural variant and/or a breakpoint of the structural variant is at a closest distance of about 34,531 base pairs from the CD273 gene.
C9.7 The method of any one of embodiments C9.4 to C9.6, wherein the distance is a linear distance.
C10. The method of any one of embodiments C1 to C9.7, wherein the structural variant comprises an ectopic portion of genomic DNA from positions 18,377,000 to 18,386,000 on chromosome 9.
C11. The method of any one of embodiments C1 to C10, wherein the structural variant comprises an ectopic portion of genomic DNA from positions 18,381,001 to 18,382,000 on chromosome 9.
C12. The method of any one of embodiments C1 to C11, wherein the adjacent location is on the p arm of chromosome 9.
C13. The method of any one of embodiments C10 to C12, wherein the ectopic portion of genomic DNA is on the p arm of chromosome 9.
C14. The method of any one of embodiments C1 to C13, wherein the cancer is a rare cancer.
C15. The method of any one of embodiment C1 to C14, wherein the cancer is kidney cancer, a cancer of the central nervous system (CNS), breast cancer, colorectal cancer, gastric cancer, lung cancer, thyroid cancer, or testicular cancer.
C16. The method of embodiment C15, wherein the cancer is a cancer of the central nervous system (CNS).
C17. The method of any one of embodiments C14 to C16, wherein the cancer is a brain cancer.
C18. The method of embodiment C17, wherein the cancer is a glioma.
C19. The method of embodiment C18, wherein the cancer is a glioblastoma.
C20. The method of embodiment C19, wherein the cancer is glioblastoma multiforme.
C21. The method of embodiment C19 or C20, wherein the cancer is pediatric glioblastoma.
C22. The method of any one of embodiments C19 to C21, wherein the glioblastoma is a newly diagnosed glioblastoma.
C23. The method of any one of embodiments C19 to C21, wherein the glioblastoma is a recurrent glioblastoma.
C24. The method of any one of embodiments C19 to C23, wherein the subject is refractory to a standard of care treatment for glioblastoma.
C25. The method of embodiment C24, wherein the standard of care treatment comprises performing and/or administering one or more of the following: surgical tumor resection, radiotherapy, positron emission tomography (PET)-guided radiotherapy, positron emission tomography (PET)-guided dose escalated radiotherapy, laser interstitial thermal therapy, stereotactic radiosurgery (SRS), hypofractionated stereotactic radiotherapy (HFSRT), Tumor-Treating Fields (TTFields), chemoradiotherapy, brachytherapy, carmustine implantable wafers, temozolomide, vincristine, interferon, bevacizumab, onartuzumab, a nitrosourea, procarbazine, enzastaurin, teniposide, cytarabine, vincristine, irinotecan, carboplatin, dasatinib, temsirolimus, erlotinib, sorafenib, veliparib, galunisertib, cediranib, vorinostat, panobinostat, dianhydrogalactitol (VAL-083) and paclitaxel poliglumex (PPX).
C26. The method of embodiment C24 or C25, wherein the standard of care treatment comprises performing and/or administering surgical tumor resection, radiotherapy and temozolomide.
C27. The method of any one of embodiments C24 to C26, wherein the standard of care treatment comprises carmustine implantable wafers.
C28. The method of any one of embodiments C24 to C27, wherein the standard of care treatment comprises administering bevacizumab.
C29. The method of any one of embodiments C24 to C28, wherein the standard of care treatment comprises administering a nitrosourea selected from among carmustine, lomustine, nimustine, nimustine hydrochloride and fotemustine.
C30. The method of any one of embodiments C24 to C29, wherein the standard of care treatment comprises administering temozolomide and bevacizumab.
C31. The method of any one of embodiments C24 to C30, wherein the standard of care treatment comprises performing and/or administering stereotactic radiosurgery (SRS) and/or hypofractionated stereotactic radiotherapy (HFSRT) and temozolomide.
C32. The method of any one of embodiment C1 to C31, wherein the structural variant comprises one or more of a translocation, inversion, insertion, deletion, and duplication.
C33. The method of any one of embodiments C1 to C32, wherein the structural variant comprises a microduplication and/or a microdeletion.
C34. The method of any one of embodiments C1 to C33, wherein determining whether the subject comprises a structural variant in the genome of the subject comprises:
   1) performing a nucleic acid analysis on a sample obtained from a subject; and
   2) detecting whether the structural variant is present or absent in the sample according to the analysis in 1).
C35. The method of embodiment C34, wherein the nucleic acid analysis in 1) comprises a method that preserves spatial-proximal contiguity information.
C36. The method of embodiment C34 or C35, wherein the nucleic acid analysis in 1) comprises one or more of polymerase chain reaction (PCR), targeted sequencing, microarray, and fluorescence in situ hybridization (FISH).
C37. The method of any one of embodiments C34 to C36, wherein the nucleic acid analysis in 1) comprises generating proximity ligated nucleic acid molecules.
C38. The method of embodiment C37, wherein the nucleic acid analysis in 1) further comprises sequencing the proximity ligated nucleic acid molecules.
C39. The method of any one of embodiments C1 to C38, wherein the subject is a human.
C40. The method of embodiment C39, wherein the subject is an adult patient.
C41. The method of embodiment C39, wherein the subject is a pediatric patient.
C42. The method of any one of embodiments C34 to C41, wherein the sample is a tissue sample, a cell sample, a blood sample, or a urine sample.
C43. The method of any one of embodiments C34 to C42, wherein the sample comprises FFPE tissue.
C44. The method of any one of embodiments C34 to C43, wherein the sample comprises frozen tissue.
C45. The method of any one of embodiments C34 to C44, wherein the sample comprises peripheral blood.
C46. The method of any one of embodiments C34 to C45, wherein the sample comprises blood obtained from bone marrow.
C47. The method of any one of embodiments C34 to C44, wherein the sample comprises cells obtained from urine.
C48. The method of any one of embodiments C34 to C47, wherein the sample comprises cell-free nucleic acid.
C49. The method of any one of embodiments C34 to C48, wherein the sample comprises one or more tumor cells.
C50. The method of any one of embodiments C34 to C49, wherein the sample comprises one or more circulating tumor cells.
C51. The method of any one of embodiments C34 to C50, wherein the sample comprises or is obtained from a solid tumor.
C52. The method of any one of embodiments C34 to C51, wherein the sample comprises or is obtained from a brain tumor.
C53. The method of embodiment C52, wherein the brain tumor is a glioblastoma.
C54. The method of any one of embodiments C1 to C53, further comprising, administering the treatment that inhibits a PD-1 receptor-mediated pathway and/or inhibits the interaction of the PD-1 receptor with PD-L1 and/or PD-L2 to the subject identified as potentially responsive to treatment with an agent that inhibits a PD-1 receptor-mediated pathway and/or inhibits the interaction of the PD-1 receptor with PD-L1 and/or PD-L2.
C55. The method of embodiment C54, wherein the treatment comprises immunotherapy.
C56. The method of any one of embodiments C1 to C55, wherein the treatment that inhibits a PD-1 receptor-mediated pathway and/or inhibits the interaction of the PD-1 receptor with PD-L1 and/or PD-L2 is selected from a treatment comprising administering one or more of atezolizumab, avelumab, balstilimab, cemiplimab, cemiplimab-rwlc, dostarlimab, dostarlimab-gxly, durvalumab, nivolumab, pembrolizumab, penpulimab, retifanlimab, sintilimab, pidilizumab, BMS-936559 (MDX-1105), AMP-224 fusion protein and MPDL33280A.
C57. The method of embodiment C56, wherein the treatment that inhibits a PD-1 receptor-mediated pathway and/or inhibits the interaction of the PD-1 receptor with PD-L1 and/or PD-L2 comprises administering pembrolizumab.
C58. The method of any one of embodiments C1 to C57, wherein the treatment that inhibits a PD-1 receptor-mediated pathway and/or inhibits the interaction of the PD-1 receptor with PD-L1 and/or PD-L2 is administered in addition to a standard of care treatment for the cancer.
C59. The method of any one of embodiments C1 to C57, wherein the treatment that inhibits a PD-1 receptor-mediated pathway and/or inhibits the interaction of the PD-1 receptor with PD-L1 and/or PD-L2 is administered instead of a standard of care treatment for the cancer.
C60. The method of any one of embodiments C1 to C59, wherein the subject is refractory to a standard of care treatment for the cancer.
C61. The method of any one of embodiments C1 to C60, further comprising predicting an outcome of a treatment that inhibits a PD-1 receptor-mediated pathway and/or inhibits the interaction of the PD-1 receptor with PD-L1 and/or PD-L2 when the structural variant is present.
D1. A method for treating a brain cancer, comprising administering a PD-1 receptor-mediated pathway inhibitor to a subject in need thereof in an amount effective for treating the brain cancer.
D2. The method of embodiment D3, wherein the cancer is a glioma.
D3. The method of embodiment D4, wherein the cancer is a glioblastoma.
D4. The method of embodiment D5, wherein the cancer is glioblastoma multiforme.
D5. The method of embodiment D3 or D4, wherein the cancer is pediatric glioblastoma.
D6. The method of any one of embodiments D3-D5, wherein the glioblastoma is a newly diagnosed glioblastoma.
D7. The method of any one of embodiments D3-D6, wherein the glioblastoma is a recurrent glioblastoma.
D8. The method of any one of embodiments D1-D7, wherein the subject is refractory to a standard of care treatment for glioblastoma.
D9. The method of any one of embodiments D1-D8, wherein the subject is a pediatric subject.
D10. The method of any one of embodiments D1-D9, wherein the PD-1 receptor-mediated pathway inhibitor inhibits the interaction of the PD-1 receptor with PD-L1 and/or PD-L2.
D11. The method of any one of embodiments D1-D10, comprising administering an immunotherapy.
D12. The method of any one of embodiments D1-D11, comprising administering one or more of atezolizumab, avelumab, balstilimab, cemiplimab, cemiplimab-rwlc, dostarlimab, dostarlimab-gxly, durvalumab, nivolumab, pembrolizumab, penpulimab, retifanlimab, sintilimab, pidilizumab, BMS-936559 (MDX-1105), AMP-224 fusion protein and MPDL33280A.
D13. The method of any one of embodiments D1-D12, comprising administering pembrolizumab.
D14. The method of any one of embodiments D1-D13, wherein the PD-1 receptor-mediated pathway inhibitor is administered in addition to a standard of care treatment for the cancer.
D15. The method of any one of embodiments D1-D13, wherein the PD-1 receptor-mediated pathway inhibitor is administered instead of a standard of care treatment for the cancer.
D16. The method of any one of embodiments D8-D15, wherein the standard of care treatment comprises performing and/or administering one or more of the following: surgical tumor resection, radiotherapy, positron emission tomography (PET)-guided radiotherapy, positron emission tomography (PET)-guided dose escalated radiotherapy, laser interstitial thermal therapy, stereotactic radiosurgery (SRS), hypofractionated stereotactic radiotherapy (HFSRT), Tumor-Treating Fields (TTFields), chemoradiotherapy, brachytherapy, carmustine implantable wafers, temozolomide, vincristine, interferon, bevacizumab, onartuzumab, a nitrosourea, procarbazine, enzastaurin, teniposide, cytarabine, vincristine, irinotecan, carboplatin, dasatinib, temsirolimus, erlotinib, sorafenib, veliparib, galunisertib, cediranib, vorinostat, panobinostat, dianhydrogalactitol (VAL-083) and paclitaxel poliglumex (PPX).

### EXAMPLES

The examples set forth below illustrate certain implementations and do not limit the technology.

### Examples

The examples set forth below illustrate certain implementations and do not limit the technology.

### Example 1: Identification of Structural Variants in Cancer Samples

In this Example, the identification of structural variants in cancer samples is described.

### HiC for FFPE

For FFPE samples, 1-10 FFPE sections of 5-10 μm thickness were subject to a HiC protocol for FFPE tissues (Arima Genomics, San Diego, CA). The FFPE samples were deparaffinized and rehydrated using one incubation with Xylene, one incubation with 100% ethanol, and one incubation with water. Following the water incubation, the deparaffinized and rehydrated tissue was incubated in Lysis Buffer (formulation below in Table 2) on ice for 20 min.

TABLE 2
Lysis Buffer

| Reagent | Stock Conc. | Units | μL/rxn | Final Conc. | Units | Master Mix |
|---|---|---|---|---|---|---|
| Tris-HCl | 1000 | mM | 1.667 | 8.333 | mM | 62.333 |
| pH 8.0 | | | | | | |
| NaCl | 1000 | mM | 1.667 | 8.333 | mM | 62.333 |
| IGEPAL | 10 | % | 3.333 | 0.167 | % | 124.667 |
| Protease | 100 | % | 33.333 | 16.667 | % | 1246.667 |
| Inhibitor | | | | | | |
| Cocktail | | | | | | |
| DI Water | | | 160.00 | | | 5984.000 |
| | | Total/rxn | 200.00 μL/rxn | | | 7480 |

Following lysis incubation, samples were pelleted, decanted, and resuspended in 20 μl of 1× Tris Buffer pH 7.4.

Then, 24 μl of Conditioning Solution (formulation below in Table 3) was added and the samples were incubated at 74° C. for 40 min.

TABLE 3
Conditioning Solution

| | Stock | | | Final | | Master |
|---|---|---|---|---|---|---|
| Reagent | Conc. | Units | μL/rxn | Conc. | Units | Mix |
| SDS | 20 | % | 1.104 | 0.920 | % | 41.290 |
| DI Water | | | 22.896 | | | 856.310 |
| | | Total/rxn | 24.000 μL/rxn | | | 897.6 |

20 μl of Stop Solution 2 (10.71% TritonX-100) was then added and the samples were incubated at 37° C. for 15 min.

After incubation in the Stop Solution, 12 μl of a Digestion Master Mix (formulation below in Table 4) was added and the samples were incubated for 1 hr at 37° C., followed by 20 min at 62° C.

TABLE 4
Digestion Master Mix

| | | | | | | |
|---|---|---|---|---|---|---|
| | Stock | | | Final | | Master |
| Reagent | Conc. | Units | μL/rxn | Conc. | Units | Mix |

| | | | | |
|---|---|---|---|---|
| NEB3.1 | 10 | x | 7.000 | 261.800 |
| Dpnll | 50 | U/μL | 1 | 37.400 |
| Hinfl | 50 | U/μL | 4 | 149.6000 |
| | | Total/rxn | 12.000 μL/rxn | 448.8 |

Then, 16 μl of a Fill-In Master Mix (formulation below in Table 5) was added and the samples were incubated for 45 min at 23° C. (room temperature).

TABLE 5
Fill-In Master Mix

| | Stock | | | Final | | Master |
|---|---|---|---|---|---|---|
| Reagent | Conc. | Units | μL/rxn | Conc. | Units | Mix |
| dCTP | 10 | mM | 0.281 | 0.176 | mM | 10.509 |
| dGTP | 10 | mM | 0.281 | 0.176 | mM | 10.509 |
| dTTP | 10 | mM | 0.281 | 0.176 | mM | 10.509 |
| Biotin-dATP | 0.4 | mM | 7.013 | 0.175 | mM | 262.286 |
| 1X NEB3.1 | 1 | X | 4.144 | 0.259 | X | 154.986 |
| Klenow | 5 | U/μL | 4.000 | 1.250 | U/μL | 149.600 |
| | | Total/rxn | 16.000 μL/rxn | | | 598.4 |

82 μl of a Ligation Master Mix (formulation below in Table 6) was then added and the samples were incubated overnight at 23° C. (room temperature).

TABLE 6
Ligation Master Mix

| Reagent | Stock Conc. | Units | μL/rxn | Final Conc. | Units | Master Mix |
|---|---|---|---|---|---|---|
| 10% TritonX-100 | 10 | % | 13.580 | 1.656 | % | 507.892 |
| BSA | 100 | X | 1.650 | 2.012 | X | 61.710 |
| Ligase Buffer | 10 | X | 16.500 | 2.012 | X | 617.100 |
| T4 DNA Ligase | | | 12.00 | | | 448.800 |
| DI Water | | | 38.270 | | | 1431.298 |
| | | Total/rxn | 82.000 μL/rxn | | | 3066.8 |

Following the ligation incubation, 16.6 μl of 5 M NaCl was added and the samples were incubated overnight at 65° C.

Then, 35.5 μl of a Reverse Crosslinking Master Mix (formulation below in Table 7) was added and the samples were incubated overnight at 55° C.

TABLE 7
Reverse Crosslinking Master Mix

| | Stock | | | Final | | Master |
|---|---|---|---|---|---|---|
| Reagent | Conc. | Units | μL/rxn | Conc. | Units | Mix |
| SDS | 20 | % | 10.500 | 2.561 | % | 261.800 |
| Proteinase | | | 25.000 | | | 935.000 |
| K | | | | | | |
| | | Total/rxn | 35.000 μL/rxn | | | 1327.7 |

Following the reverse crosslinking incubation, DNA was purified using SPRI beads and then sonicated/sheared. DNA was size selected for fragments 200-600 bp in length using SPRI beads. Biotinylated DNA was enriched using Streptavidin beads, and on-bead DNA fragments were converted into adapter ligated Illumina sequencing libraries using reagents from the SWIFT ACCEL-NGS 2S Plus DNA Library Kit (Swift Biosciences/IDT).

Then, adapter ligated and bead-bound DNA was PCR amplified using reagents from KAPA, and the resulting PCR-amplified DNA was purified using SPRI beads. For samples subject to Capture-HiC, sufficient PCR cycles were used in order to obtain at least 500 ng (optimally 1500 ng) of DNA (the minimum amount of DNA used for probe hybridization in the Capture-HiC protocol). HiC libraries were subject to shallow sequencing QC on an Illumina MINISEQ. HiC libraries were subject to deep NGS on either Illumina HISEQ or NOVASEQ instruments.

### HiC for Blood

The HiC protocol for blood (Arima Genomics, San Diego, CA) matches that of FFPE protocol described above, except for the following differences.

Blood samples are not already fixed and then are not paraffin embedded. Therefore, the first step for blood is to crosslink blood cells using 2% formaldehyde for 10 min, quench crosslinking using a final concentration of 125 mM Glycine, and then begin HiC with the Lysis Step (see above).

The blood protocol differs from FFPE in the Conditioning Solution step, where Conditioning Solution for blood is added at 62° C. for 10 min. The blood protocol also differs from FFPE in the Ligation step, where Ligation reaction is 15 min instead of overnight. The blood protocol also differs from FFPE after Ligation but before DNA purification, in that a single Reverse Crosslinking master mix containing Proteinase K, NaCl, and SDS is added to the sample and it is incubated at 55° C. for 30 min, then 68° C. for 90 min, and then purified using SPRI beads.

The remainder of the protocol, including DNA shearing, size selection, library prep, PCR and Capture-HiC (below) is the same between blood and FFPE.

### Capture-HiC

First, 1500 ng of amplified HiC library was “pre-cleared” in order to remove residual biotinylated DNA. This was done by negative selection—the 1500 ng of amplified HiC library was combined with streptavidin beads, and the unbound DNA fraction was carried forward and the bound fraction was discarded.

The now pre-cleared amplified HiC library was then subject to Capture Enrichment, consisting of a) hybridization, b) capture; and c) amplification; according to the Agilent SURESELECT XTHS reagents and standard protocol. Capture targets/probes were custom-designed by Arima, using the Agilent SUREDESIGN software suite (details below). Following Capture Enrichment, Capture-HiC libraries were shallow sequenced on a MINISEQ or more deeply sequenced on an Illumina HISEQ.

### Capture Probe Design

A list of unique genes was compiled from the following sources:
NYU GenomePACT Panel
NYU Fusion SEQ'r Panel
ArcherDx VariantPlex Myeloid Panel
ArcherDx Pan Heme Panel
Stanford STAMP Heme Panel
ArcherDx Pan Solid Tumor
ArcherDx VariantPlex Solid Tumor
Childrens' Hospital of Philadelphia (CHOP) Comprehensive Tumor and Fusion Panel
Agilent All-in-One Solid Tumor Panel
Agilent ClearSeq Comprehensive Cancer Panel
Foundation Medicine Foundation One CDx Panel
Stanford STAMP Solid Tumor Panel
Stanford STAMP Fusion Panel

These genes were then cross-referenced to the Ensembl data base, with 885 total genes collected (see Table 8 below). The exon coordinates were then located for all 885 genes, as well as the HiC restriction enzyme cut sites (Arima Genomics, San Diego, CA) within and directly flanking the exons. To define the target capture regions, the sequences within 350 bp from restriction enzyme cut sites were identified. For cut sites flanking the exons, the “inward” 350 bp (the 350 bp in the direction of the exon) was targeted. For this probe design, the cut sites were: \hat{ }GATC and G\hat{ }ANTC (where \hat{ } is the cut site on the positive strand, and “N” can be any of the 4 genomic bases, A, C, G, T). Collectively, this approach identified a set of coordinates in and around exons of genes of interest. These coordinates were then uploaded into the Agilent SUREDESIGN™ Software Suite for the design of individual probe sequences. Probe design was carried out using some custom parameters, including 1× tiling density, moderate stringency repeat masking, and optimized performance boosting. The probes were designed against the HG38 human reference genome. The total size of the target region was 12.075 Mb and following probe design 92.79449% (11.483 Mb) was covered by probes. In total, 335,242 probes were designed.

TABLE 8
Oncopanel genes

| | | | | |
|---|---|---|---|---|
| ABCB1 | CXCR4 | HOXA10 | NELL2 | RPS15 |
| ABCC2 | CXXC5 | HOXA9 | NF1 | RPS6KA2 |
| ABL1 | CYB5R2 | HOXB13 | NF2 | RPS6KB1 |
| ABL2 | CYLD | HRAS | NFATC2 | RPTOR |
| ABRAXAS1 | CYP17A1 | HSD3B1 | NFE2L2 | RRM1 |
| ACTG1 | CYP19A1 | HSP90AA1 | NFIB | RSPO2 |
| ACVR1 | CYP2A6 | HSP90AB1 | NFKB1 | RSPO3 |
| ACVR1B | CYP2B6 | ID3 | NFKB2 | RUNX1 |
| ACVR2A | CYP2C19 | ID4 | NFKBIA | RUNX1T1 |
| ADAMTS20 | CYP2C9 | IDH1 | NFKBIE | RXRA |
| ADGRA2 | CYP2D6 | IDH2 | NIN | RXRB |
| ADGRB3 | DAXX | IGF1R | NKX2-1 | RXRG |
| ADGRF5 | DCC | IGF2 | NLRP1 | S1PR2 |
| ADGRL3 | DCK | IGF2R | NME1 | SAMD9 |
| AFDN | DDB2 | IGHA1 | NOTCH1 | SBDS |
| AFF1 | DDIT3 | IGHA2 | NOTCH2 | SDC4 |
| AFF3 | DDR1 | IGHG1 | NOTCH3 | SDHA |
| AICDA | DDR2 | IGHG2 | NOTCH4 | SDHB |
| AKAP9 | DDX3X | IGHG3 | NPM1 | SDHC |
| AKT1 | DDX41 | IGHG4 | NR4A3 | SDHD |
| AKT2 | DEK | IGHJ1 | NRAS | SEMA6A |
| AKT3 | DENND3 | IGHJ2 | NRG1 | SERPINA9 |
| ALK | DHX15 | IGHJ3 | NSD1 | SETBP1 |
| ALOX12B | DICER1 | IGHJ4 | NSD2 | SETD2 |
| AMER1 | DIS3 | IGHJ5 | NSD3 | SETD5 |
| ANKRD24 | DLEU1 | IGHJ6 | NT5C2 | SF3B1 |
| ANKRD26 | DNAH9 | IGHM | NTRK1 | SGK1 |
| APC | DNAJB1 | IKBKB | NTRK2 | SH2B3 |
| APLNR | DNM2 | IKBKE | NTRK3 | SH2D1A |
| AR | DNMT3A | IKZF1 | NUMA1 | SH3BP5 |
| ARAF | DNMT3B | IKZF2 | NUMBL | SHH |
| ARFGAP3 | DNTT | IKZF3 | NUP214 | SHOC2 |
| ARFRP1 | DOT1L | IL16 | NUP93 | SLC22A1 |
| ARHGAP26 | DPH3 | IL2 | NUP98 | SLC22A2 |
| ARHGAP6 | DPYD | IL21R | NUTM1 | SLC29A1 |
| ARID1A | DROSHA | IL2RA | NUTM2A | SLC31A1 |
| ARID1B | DST | IL2RB | OGA | SLC34A2 |
| ARID2 | DUSP22 | IL2RG | P2RY8 | SLC45A3 |
| ARNT | E2F2 | IL3 | PAG1 | SLCO1B1 |
| ASB13 | EBF1 | IL3RA | PAICS | SMAD2 |
| ASH1L | EED | IL6ST | PAK3 | SMAD4 |
| ASPSCR1 | EGF | IL7R | PALB2 | SMARCA4 |
| ASXL1 | EGFR | ING4 | PARP1 | SMARCB1 |
| ATF1 | EGR1 | INHBA | PARP2 | SMARCE1 |
| ATM | EIF4A1 | INPP4B | PARP3 | SMC1A |
| ATR | EML4 | INSR | PAX3 | SMC3 |
| ATRX | EMSY | IRAG2 | PAX5 | SMO |
| AURKA | ENTPD1 | IRF2 | PAX7 | SMUG1 |
| AURKB | EP300 | IRF4 | PAX8 | SNCAIP |
| AURKC | EP400 | IRF8 | PBRM1 | SNX31 |
| AUTS2 | EPC1 | IRS2 | PBX1 | SOCS1 |
| AXIN1 | EPCAM | ITGA10 | PCBP1 | SOCS3 |
| AXL | EPHA2 | ITGA9 | PCDHAC2 | SOS1 |
| B2M | EPHA3 | ITGB2 | PCLAF | SOX10 |
| BAP1 | EPHA5 | ITGB3 | PDCD1 | SOX11 |
| BARD1 | EPHA7 | ITK | PDCD1LG2 | SOX2 |
| BATF3 | EPHB1 | ITPKB | PDE4DIP | SOX9 |
| BAX | EPHB4 | JAK1 | PDGFB | SP140 |
| BCL10 | EPHB6 | JAK2 | PDGFD | SPEN |
| BCL11A | EPOR | JAK3 | PDGFRA | SPI1 |
| BCL11B | ERBB2 | JARID2 | PDGFRB | SPOP |
| BCL2 | ERBB3 | JAZF1 | PDK1 | SPRED1 |
| BCL2A1 | ERBB4 | JMJD1C | PER1 | SPTA1 |
| BCL2L1 | ERCC1 | JUN | PGAP3 | SRC |
| BCL2L2 | ERCC2 | KAT6A | PHF1 | SRSF2 |
| BCL3 | ERCC3 | KAT6B | PHF6 | SS18 |
| BCL6 | ERCC4 | KDM5A | PHKB | SS18L1 |
| BCL9 | ERCC5 | KDM5C | PHLPP2 | SSX1 |
| BCOR | ERG | KDM6A | PHOX2B | SSX2 |
| BCORL1 | ERRFI1 | KDR | PICALM | SSX4 |
| BCR | ESR1 | KEAP1 | PIGA | STAG2 |
| BEND2 | ESR2 | KEL | PIK3C2B | STAT1 |
| BIRC2 | ESRRA | KIT | PIK3C2G | STAT3 |
| BIRC3 | ETNK1 | KLF2 | PIK3C3 | STAT4 |
| BIRC5 | ETS1 | KLF6 | PIK3CA | STAT5B |
| BLM | ETV1 | KLHL6 | PIK3CB | STAT6 |
| BLNK | ETV4 | KMT2A | PIK3CD | STIL |
| BMF | ETV5 | KMT2B | PIK3CG | STK11 |
| BMP7 | ETV6 | KMT2C | PIK3R1 | STK36 |
| BMPR1A | EWSR1 | KMT2D | PIK3R2 | STRBP |
| BOD1L1 | EXOC2 | KNL1 | PIM1 | STX11 |
| BRAF | EXT1 | KRAS | PIM2 | SUFU |
| BRCA1 | EXT2 | LAMA2 | PKD1L2 | SUZ12 |
| BRCA2 | EZH1 | LAMP1 | PKHD1 | SYK |
| BRD3 | EZH2 | LCK | PKN1 | SYNE1 |
| BRD4 | EZR | LIFR | PLAG1 | SYT1 |
| BRINP3 | FAM216A | LIMD1 | PLCG1 | TAF1 |
| BRIP1 | FANCA | LMO1 | PLCG2 | TAF15 |
| BTG1 | FANCC | LMO2 | PLEKHG5 | TAF1L |
| BTK | FANCD2 | LPP | PLEKHS1 | TAL1 |
| BUB1B | FANCE | LRP1B | PML | TAS2R38 |
| CACNA1E | FANCF | LTF | PMS1 | TBX22 |
| CALR | FANCG | LTK | PMS2 | TBX3 |
| CAMTA1 | FANCL | LUC7L2 | POLD1 | TCF12 |
| CARD11 | FAS | LYL1 | POLE | TCF3 |
| CASP8 | FBXW4 | LYN | POT1 | TCF7L1 |
| CBFA2T3 | FBXW7 | LZTR1 | POU5F1 | TCF7L2 |
| CBFB | FGF1 | LZTS1 | PPARG | TCL1A |
| CBL | FGF10 | MAF | PPAT | TEK |
| CBLB | FGF12 | MAFB | PPM1D | TENT5C |
| CBLC | FGF14 | MAGEA1 | PPP2R1A | TERC |
| CCDC170 | FGF19 | MAGI1 | PPP2R2A | TERT |
| CCDC50 | FGF23 | MAL | PPP6C | TET1 |
| CCN6 | FGF3 | MALT1 | PRCC | TET2 |
| CCNB3 | FGF4 | MAML2 | PRDM1 | TET3 |
| CCND1 | FGF6 | MAML3 | PRDM10 | TFE3 |
| CCND2 | FGFR1 | MAMLD1 | PRDM16 | TFEB |
| CCND3 | FGFR2 | MAP2K1 | PREX2 | TFG |
| CCNE1 | FGFR3 | MAP2K2 | PRKACA | TGFB1 |
| CCR4 | FGFR4 | MAP2K4 | PRKACB | TGFBR2 |
| CD22 | FGR | MAP3K1 | PRKAR1A | TGFBR3 |
| CD274 | FH | MAP3K13 | PRKAR2B | TGM7 |
| CD28 | FIP1L1 | MAP3K7 | PRKCA | THADA |
| CD44 | FLCN | MAPK1 | PRKCB | THBS1 |
| CD58 | FLI1 | MAPK8 | PRKCD | TIMP3 |
| CD70 | FLT1 | MARK1 | PRKCI | TIPARP |
| CD74 | FLT3 | MARK4 | PRKD1 | TLR2 |
| CD79A | FLT4 | MAST1 | PRKD2 | TLR4 |
| CD79B | FN1 | MAST2 | PRKD3 | TLX1 |
| CD83 | FOS | MBD1 | PRKDC | TLX3 |
| CDA | FOSB | MBTD1 | PRPF8 | TMEM216 |
| CDC25A | FOXA1 | MCL1 | PSIP1 | TMPRSS2 |
| CDC25C | FOXL2 | MDM2 | PSMB1 | TNFAIP3 |
| CDC73 | FOXO1 | MDM4 | PSMB2 | TNFRSF13B |
| CDH1 | FOXO3 | MEAF6 | PSMB5 | TNFRSF14 |
| CDH11 | FOXO4 | MECOM | PSMD1 | TNFRSF1A |
| CDH2 | FOXP1 | MED12 | PSMD2 | TNFRSF1B |
| CDH20 | FOXP4 | MED13 | PTCH1 | TNFSF4 |
| CDH23 | FOXR2 | MEF2B | PTEN | TNK2 |
| CDH5 | FSTL5 | MEN1 | PTGS2 | TOP1 |
| CDK12 | FUBP1 | MERTK | PTK2B | TP53 |
| CDK4 | FUS | MET | PTPN1 | TP63 |
| CDK6 | FUT8 | MITF | PTPN11 | TPM3 |
| CDK8 | FYN | MKNK1 | PTPRD | TPR |
| CDKN1A | FZR1 | MLC1 | PTPRO | TRAF3 |
| CDKN1B | G6PD | MLF1 | PTPRT | TRIM24 |
| CDKN2A | GABRA6 | MLH1 | PYCR1 | TRIM33 |
| CDKN2B | GATA1 | MLH3 | QKI | TRIP11 |
| CDKN2C | GATA2 | MLLT1 | RAB29 | TRRAP |
| CEBPA | GATA3 | MLLT10 | RAC1 | TSC1 |
| CEBPD | GATA6 | MME | RAD21 | TSC2 |
| CEBPE | GDNF | MMP2 | RAD50 | TSHR |
| CEBPG | GID4 | MN1 | RAD51 | TSLP |
| CHD1 | GLI1 | MNX1 | RAD51B | TYK2 |
| CHD2 | GLIS2 | MPL | RAD51C | TYRO3 |
| CHD4 | GNA11 | MRE11 | RAD51D | U2AF1 |
| CHD5 | GNA13 | MRTFA | RAD52 | U2AF2 |
| CHD7 | GNAI3 | MRTFB | RAD54L | UBR5 |
| CHEK1 | GNAQ | MSH2 | RAF1 | UGT1A1 |
| CHEK2 | GNAS | MSH3 | RAG1 | UMODL1 |
| CHIC2 | GNB1 | MSH6 | RAG2 | USP6 |
| CIC | GPS2 | MSMB | RALGDS | USP9X |
| CIITA | GRB7 | MST1R | RANBP1 | VAV1 |
| CILK1 | GRIN2A | MTAP | RARA | VEGFA |
| CKS1B | GRM3 | MTOR | RARB | VGLL2 |
| CMPK1 | GRM8 | MTR | RARG | VGLL3 |
| COL1A1 | GSK3B | MTRR | RB1 | VHL |
| CRBN | GSTP1 | MUC1 | RBBP6 | WAS |
| CREB1 | GUCY1A2 | MUSK | RBM10 | WRN |
| CREB3L2 | H1-2 | MUTYH | RBM15 | WT1 |
| CREBBP | H1-3 | MYB | RECQL4 | WWWTR1 |
| CRKL | H1-4 | MYBL1 | REL | XPA |
| CRLF2 | H1-5 | MYC | RELA | XPC |
| CRTC1 | H2AC6 | MYCL | RET | XPO1 |
| CSF1 | H3-3A | MYCN | RHEB | XRCC2 |
| CSF1R | H3-3B | MYD88 | RHOA | YAP1 |
| CSF3R | H3C14 | MYH11 | RHOH | YES1 |
| CSMD3 | H3C2 | MYH9 | RICTOR | YWHAE |
| CSNK2B | HCAR1 | MYOD1 | RIT1 | ZCCHC7 |
| CTCF | HDAC1 | NAB2 | RNASEL | ZMYM2 |
| CTDNEP1 | HGF | NBN | RNF2 | ZMYM3 |
| CTLA4 | HIF1A | NCOA1 | RNF213 | ZNF217 |
| CTNNA1 | HLF | NCOA2 | RNF43 | ZNF384 |
| CTNNB1 | HMGA2 | NCOA3 | ROS1 | ZNF521 |
| CUL3 | HNF1A | NCOA4 | RPL22 | ZNF703 |
| CUL4A | HNRNPK | NCOR2 | RPN1 | ZRSR2 |
| CUX1 | HOOK3 | NEK6 | RPS14 | ZSWIM4 |

### HiC Data Analysis

To identify structural variants, raw HiC read-pairs were mapped to the human reference (hg38) and deduplicated. Mapped and deduplicated read pairs were then analyzed using the HiC-BREAKFINDER software (Dixon, Nature Genetics, 2018) to call structural variants.

For data visualization, HiC read-pairs were analyzed using the JUICER software, which outputs a “.hic” file that can be uploaded into the desktop JUICEBOX software for visualization of HiC heatmaps. Visual inspection, along with the structural variant calls from HiC-BREAKFINDER, were used to approximate the structural variant breakpoints from HiC analysis.

### Capture-HiC Data Preliminary Analysis

To identify structural variants, raw Capture-HiC read-pairs were mapped to the human reference (hg38) and deduplicated. Then, the genome was binned into different size genomic bins (e.g. 1 Mb, 50 kb, 1 kb), and then the total observed HiC read-pairs was summed between the gene of interest and every other bin in the genome. Each pair was tested (i.e., the number of counts between the gene of interest and Bin X) for statistical significance, modeled against a null distribution from non-tumor Capture-HiC data, and corrected for multiple testing. The output of this analysis are bins of the genome with statistically significant observed interactions with the gene of interest. The premise is that the gene within the bin(s) of highest statistical significance is involved in a structural variant with the gene of interest.

For data visualization, the observed read counts between a gene of interest and all other genomic bins can be represented as a “Manhattan Plot”. Data can also be visualized in the IGV browser, but portraying only the read-pairs with at least 1 end mapping to the gene of interest.

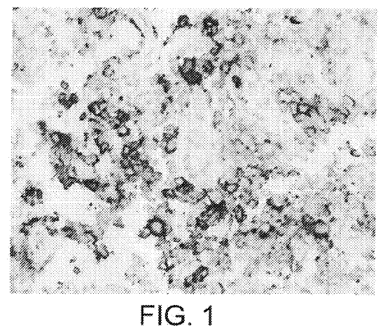

FIG. 1 shows an immunohistochemistry stain using anti-PD-L1 antibody in glioblastoma tumor cells. A proximity fusion involving PD-L1 was discovered using one embodiment of the spatial-proximal contiguity assays described herein. The expression of PD-L1 in the tumor tissue shown by the antibody stain indicates that the tumor cells are not as susceptible to the immune system as tumor cells without PD-L1 expression would be. Treatment with drugs that block PD-L1 (or the broader PD-1 receptor-mediated pathway) would allow tumor cells to be susceptible to the patient's T-cells.

### Structural Variant Identified

Table 9 below shows the structural variant adjacent to the CD274 gene that was identified by methods described herein. Samples were classified as having undiagnosed tumors/cancers with no clear with no known tumor driver (e.g., oncogene, such as the CD274 gene) as assessed by standard cytogenetic/molecular testing (i.e., chromosomal karyotyping, a FISH panel, DNA microarray, and a cancer next generation sequencing (NGS) panel).

TABLE 9

| | |
|---|---|
| Tumor type | Glioblastoma |
| Partner 1 type | Intergenic break |
| Approximate breakpoint | chr9: 5,475,001-5,476,000 |
| coordinate window 1A | |
| Approximate breakpoint | chr9: 5,471,000-5,480,000 |
| coordinate window 1B | |
| Relevant oncogene | PD-L1 (CD274) |
| | PD-L2 (CD273) |
| Gene 5′ | PD-L1 (CD274): chr9: 5,450,542 |
| | PD-L2 (CD273): chr9: 5,510,531 |
| Gene 3′ | PD-L1 (CD274): chr9: 5,470,554 |
| | PD-L2 (CD273): chr9: 5,571,282 |
| Linear distance to 5′ (bp) | PD-L1 (CD274): 24,459 |
| | PD-L2 (CD273): 34,531 |
| Closest distance to gene body (bp) | PD-L1 (CD274): 4,447 |
| | PD-L2 (CD273): 34,531 |
| Partner 2 gene or intergenic | Intergenic |
| Relevant oncogene | N/A |
| Gene 5′ | N/A |
| Gene 3′ | N/A |
| Linear distance to 5′ (bp) | N/A |
| Closest distance to gene body (bp) | N/A |
| Approximate partner breakpoint | chr9: 18,381,001-18,382,000 |
| coordinate window 2A | |
| Approximate partner breakpoint | chr9: 18,377,000-18,386,000 |
| coordinate window 2B | |
| Capture or genome-wide | Genome-wide |

### Example 2: Treatment of Subject Having Glioblastoma Multiforme with a PD-1 Inhibitor

In this Example, a method of treating a subject having glioblastoma multiforme with a PD-1 inhibitor is described. A subject with newly diagnosed glioblastoma multiforme and undergoing a standard of care treatment, such as maximum safe surgical resection followed by concurrent administration of temozolomide (TMZ) (75 mg/m²/day for 6 weeks) and radiotherapy (RT) (60 Gy in 30 fractions) and then six maintenance cycles of TMZ (150-200 mg/m²/day for the first 5 days of a 28-day cycle) is determined to be refractory to the treatment, as measured by the occurrence of disease progression during or following one or more cycles of treatment. The subject is then either co-administered a PD-1 inhibitor, such as pembrolizumab, or the standard of care treatment is discontinued, and the subject is treated with the PD-1 inhibitor alone. An example of a standard of care dosage for pembrolizumab, for example, is 200 mg of pembrolizumab every 3 weeks. Responsiveness to the treatment is measured by monitoring whether there is a decrease in tumor volume over the course of administration of the treatment.

The entirety of each patent, patent application, publication and document referenced herein is incorporated by reference, to the extent permitted by law. Citations of patents, patent applications, publications and documents are not an admission that any of the foregoing is pertinent prior art, nor does it constitute any admission as to the contents or date of these publications or documents. Their citation is not an indication of a search for relevant disclosures. All statements regarding the date(s) or contents of the documents are based on available information and is not an admission as to their accuracy or correctness.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein. Unless otherwise noted, technical terms are used according to conventional usage. Definitions of common terms in molecular biology may be found in Benjamin Lewin's Genes XII, published by Jones & Bartlett Learning, 2017 (ISBN-10:1284104494) and Joseph Jez (ed), Encyclopedia of Biological Chemistry, published by Elsevier, 2021 (ISBN 9780128194607).

Each of the terms “comprising,” “consisting essentially of,” and “consisting of” may be replaced with either of the other two terms. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%; e.g., a weight of “about 100 grams” can include a weight between 90 grams and 110 grams). Use of the term “about” at the beginning of a listing of values modifies each of the values (e.g., “about 1, 2 and 3” refers to “about 1, about 2 and about 3”). When a listing of values is described the listing includes all intermediate values and all fractional values thereof (e.g., the listing of values “80%, 85% or 90%” includes the intermediate value 86% and the fractional value 86.4%). When a listing of values is followed by the term “or more,” the term “or more” applies to each of the values listed (e.g., the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (e.g., the listing of “80%, 90% or 95%” includes ranges of “80% to 90%,” “80% to 95%” and “90% to 95%”).

Certain implementations of the technology are set forth in the claim(s) that follow(s).

What is claimed is:

1. A method of treating a subject that has, or is suspected of having, cancer, the method comprising:
a) identifying and/or selecting a subject comprising a structural variant in the genome of the subject, wherein the location of the structural variant is adjacent to the CD274 and/or the CD273 gene; and
b) if the subject has cancer, treating the subject so identified and/or selected with a treatment that inhibits a PD-1 receptor-mediated pathway and/or inhibits the interaction of the PD-1 receptor with PD-L1 and/or PD-L2.

2. The method of claim 1, wherein identifying and/or selecting a subject comprising a structural variant in the genome of the subject comprises:
a) performing a nucleic acid analysis on a sample obtained from a subject; and
b) detecting whether the structural variant is present or absent in the sample according to the analysis in a).

3. The method of claim 2, wherein the nucleic acid analysis in a) comprises a method that preserves spatial-proximal contiguity information.

4. The method of claim 1, wherein the cancer is kidney cancer, a cancer of the central nervous system (CNS), breast cancer, colorectal cancer, gastric cancer, lung cancer, thyroid cancer, or testicular cancer.

5. The method of claim 4, wherein the cancer is pediatric glioblastoma.

6. The method claim 1, wherein the treatment that inhibits a PD-1 receptor-mediated pathway and/or inhibits the interaction of the PD-1 receptor with PD-L1 and/or PD-L2 is selected from a treatment comprising administering one or more of atezolizumab, avelumab, balstilimab, cemiplimab, cemiplimab-rwlc, dostarlimab, dostarlimab-gxly, durvalumab, nivolumab, pembrolizumab, penpulimab, retifanlimab, sintilimab, pidilizumab, BMS-936559 (MDX-1105), AMP-224 fusion protein and MPDL33280A.

7. The method of claim 4, wherein the subject has brain cancer and further comprising administering a PD-1 receptor-mediated pathway inhibitor to the subject in need thereof in an amount effective for treating the brain cancer.

8. A method of selecting a subject having cancer for treatment with an agent that inhibits a PD-1 receptor-mediated pathway and/or inhibits the interaction of the PD-1 receptor with PD-L1 and/or PD-L2, the method comprising:
a) determining whether the subject comprises a structural variant in the genome of the subject, wherein the location of the structural variant, or a breakpoint of the structural variant, is adjacent to the CD274 gene and/or CD273 gene; and
b) if the structural variant, or a breakpoint of the structural variant, is identified in a), selecting the subject for treatment with an agent that inhibits a PD-1 receptor-mediated pathway and/or inhibits the interaction of the PD-1 receptor with PD-L1 and/or PD-L2.

9. The method of claim 8, wherein determining whether the subject comprises a structural variant in the genome of the subject comprises:
a) performing a nucleic acid analysis on a sample obtained from a subject; and
b) detecting whether the structural variant is present or absent in the sample according to the analysis in a).

10. The method of claim 9, wherein the nucleic acid analysis in a) comprises a method that preserves spatial-proximal contiguity information.

11. The method of claim 8, wherein the cancer is kidney cancer, a cancer of the central nervous system (CNS), breast cancer, colorectal cancer, gastric cancer, lung cancer, thyroid cancer, or testicular cancer.

12. The method of claim 11, wherein the cancer is pediatric glioblastoma.

13. The method claim 8, wherein the treatment that inhibits a PD-1 receptor-mediated pathway and/or inhibits the interaction of the PD-1 receptor with PD-L1 and/or PD-L2 is selected from a treatment comprising administering one or more of atezolizumab, avelumab, balstilimab, cemiplimab, cemiplimab-rwlc, dostarlimab, dostarlimab-gxly, durvalumab, nivolumab, pembrolizumab, penpulimab, retifanlimab, sintilimab, pidilizumab, BMS-936559 (MDX-1105), AMP-224 fusion protein and MPDL33280A.

14. The method of claim 11, wherein the subject has brain cancer and further comprising administering a PD-1 receptor-mediated pathway inhibitor to the subject in need thereof in an amount effective for treating the brain cancer.

15. A method of screening a subject having cancer for potential responsiveness to treatment with an agent that inhibits a PD-1 receptor-mediated pathway and/or inhibits the interaction of the PD-1 receptor with PD-L1 and/or PD-L2, the method comprising:
a) determining whether the subject comprises a structural variant in the genome of the subject, wherein the location of the structural variant, or a breakpoint of the structural variant, is adjacent to the CD274 gene and/or CD273 gene; and
b) if the structural variant, or a breakpoint of the structural variant, is identified in a), identifying the subject as potentially responsive to treatment with an agent that inhibits a PD-1 receptor-mediated pathway and/or inhibits the interaction of the PD-1 receptor with PD-L1 and/or PD-L2.

16. The method of claim 15, wherein determining whether the subject comprises a structural variant in the genome of the subject comprises:
a) performing a nucleic acid analysis on a sample obtained from a subject; and
b) detecting whether the structural variant is present or absent in the sample according to the analysis in a).

17. The method of claim 16, wherein the nucleic acid analysis in a) comprises a method that preserves spatial-proximal contiguity information.

18. The method of claim 15, wherein the cancer is kidney cancer, a cancer of the central nervous system (CNS), breast cancer, colorectal cancer, gastric cancer, lung cancer, thyroid cancer, or testicular cancer.

19. The method of claim 18, wherein the cancer is pediatric glioblastoma.

20. The method claim 15, wherein the treatment that inhibits a PD-1 receptor-mediated pathway and/or inhibits the interaction of the PD-1 receptor with PD-L1 and/or PD-L2 is selected from a treatment comprising administering one or more of atezolizumab, avelumab, balstilimab, cemiplimab, cemiplimab-rwlc, dostarlimab, dostarlimab-gxly, durvalumab, nivolumab, pembrolizumab, penpulimab, retifanlimab, sintilimab, pidilizumab, BMS-936559 (MDX-1105), AMP-224 fusion protein and MPDL33280A.
